(19) United States
(12) Patent Application Publication
(10) Pub. No.: US 2026/0232223 A1
(43) Pub. Date: Aug. 13, 2026

(54) DATA PROCESSING DEVICE, GAIT MEASUREMENT SYSTEM, DATA PROCESSING METHOD, AND RECORDING MEDIUM

(71) Applicant: NEC Corporation, Tokyo (JP)

(72) Inventor: Hiroshi KAJITANI, Tokyo (JP)

(73) Assignee: NEC Corporation, Tokyo (JP)

(21) Appl. No.: 19/419,346

(22) Filed: Dec. 15, 2025

(30) Foreign Application Priority Data
Feb. 10, 2025 (JP) 2025-019740

(51) Int. Cl. A61B 5/11; A61B 5/00

(52) U.S. Cl. CPC A61B 5/112; A61B 5/6807

(57) ABSTRACT

Provided is a data processing device including an acquisition unit that acquires sensor data including acceleration data, angular velocity data, and magnetic data measured by a sensor mounted on footwear of a user, a rejection unit that rejects abnormality data included in the sensor data according to a variation in an azimuth angle of the sensor calculated by using the magnetic data, and an output unit that outputs the sensor data from which the abnormality data has been excluded.

This application is based upon and claims the benefit of priority from Japanese patent application No. 2025-019740, filed on February 10, 2025, the disclosure of which is incorporated herein in its entirety by reference.

### TECHNICAL FIELD

The present disclosure relates to a data processing device, a gait measurement system, a data processing method, and a program.

### BACKGROUND ART

With growing interest in healthcare, services that provide information according to gait have attracted attention. For example, a technique for analyzing gait by using sensor data measured by a sensor mounted on footwear such as shoes has been developed. In time-series data of the sensor data, a feature related to the physical condition appears. If health condition can be estimated by the feature related to physical condition, it is possible to perform early detection and prevention of diseases.

PTL 1 (JP 2018-068669 A) discloses a system that provides advice on correction of exercise, discrimination of physical condition, and detection and prevention of injuries and failures. The system of PTL 1 includes a sensor module and an information distribution terminal. The sensor module includes an attachment unit, an acceleration sensor, a gyro sensor, a storage unit, and a communication module. The information distribution terminal calculates a first difference between exercise information of a user and predetermined reference data. The information distribution terminal calculates a second difference between the exercise information of the user and an average value of past cumulative data of the user. The information distribution terminal generates analysis information based on the first difference or the second difference.

In the method of PTL 1, it is not possible to exclude abnormality data according to a sudden change in movement with respect to a variation peculiar to walking, such as a direction change in walking and disturbance in gait. Therefore, the method of PTL 1 has a problem that it takes time to stabilize and converge sensor data. In order to enable accurate gait analysis, it is required to effectively exclude abnormality data included in sensor data measured according to movement of a foot.

An object of the present disclosure is to provide a data processing device, a gait measurement system, a data processing method, and a program that enable accurate gait analysis by excluding abnormality data included in sensor data measured according to movement of a foot.

### SUMMARY

According to an aspect of the present disclosure, a data processing device includes an acquisition unit that acquires sensor data including acceleration data, angular velocity data, and magnetic data measured by a sensor mounted on footwear of a user, a rejection unit that rejects abnormality data included in the sensor data according to a variation in an azimuth angle of the sensor calculated by using the magnetic data, and an output unit that outputs the sensor data from which the abnormality data has been excluded.

According to another aspect of the present disclosure, data processing includes, by a computer, acquiring sensor data including acceleration data, angular velocity data, and magnetic data measured by a sensor mounted on footwear of a user, rejecting abnormality data included in the sensor data according to a variation in an azimuth angle of the sensor calculated by using the magnetic data, and outputting the sensor data from which the abnormality data has been excluded.

According to still another aspect of the present disclosure, a program causes a computer to execute a process including acquiring sensor data including acceleration data, angular velocity data, and magnetic data measured by a sensor mounted on footwear of a user, rejecting abnormality data included in the sensor data according to a variation in an azimuth angle of the sensor calculated by using the magnetic data, and outputting the sensor data from which the abnormality data has been excluded.

According to the present disclosure, it is possible to provide a data processing device, a gait measurement system, a data processing method, and a program that enable accurate gait analysis by excluding abnormality data included in sensor data measured according to movement of a foot.

### BRIEF DESCRIPTION OF THE DRAWINGS

FIG. 1 is a block diagram illustrating a configuration of a gait measurement system in the present disclosure;

FIG. 2 is a block diagram illustrating an example of a configuration of a measurement device in the present disclosure;

FIG. 3 is a conceptual diagram illustrating an example in which the measurement device in the present disclosure is disposed in a shoe;

FIG. 4 is a conceptual diagram for describing a local coordinate system and a world coordinate system in the present disclosure;

FIG. 5 is a conceptual diagram for describing a human body plane set for a human body;

FIG. 6 is a block diagram illustrating an example of a configuration of a data processing device in the present disclosure;

FIG. 7 is a conceptual diagram for describing one gait cycle with a right foot as a reference;

FIG. 8 illustrates an example of a table in which values of gait parameters calculated by the data processing device included in the gait measurement system in the present disclosure are collected;

FIG. 9 illustrates another example of the table in which the values of the gait parameters calculated by the data processing device included in the gait measurement system in the present disclosure are collected;

FIG. 10 is a flowchart illustrating an example of an operation of the data processing device in the present disclosure;

FIG. 11 is a block diagram illustrating an example of a configuration of a data processing device in the present disclosure;

FIG. 12 is a flowchart illustrating an example of an operation of the data processing device in the present disclosure;

FIG. 13 is a block diagram illustrating a configuration of a gait measurement system in the present disclosure;

FIG. 14 is a block diagram illustrating an example of a configuration of a gait evaluation device in the present disclosure;

FIG. 15 illustrates a display example of an evaluation result output from the gait evaluation device included in the gait measurement system in the present disclosure;

FIG. 16 illustrates another display example of the evaluation result output from the gait evaluation device included in the gait measurement system in the present disclosure;

FIG. 17 is a flowchart illustrating an example of an operation of the gait evaluation device in the present disclosure;

FIG. 18 is a block diagram illustrating an example of a configuration of a data processing device in the present disclosure;

FIG. 19 is a flowchart illustrating an example of an operation of the data processing device in the present disclosure; and

FIG. 20 is a block diagram illustrating an example of a hardware configuration that executes control and processing in the present disclosure.

### EXAMPLE EMBODIMENT

Hereinafter, modes for carrying out the present disclosure will be described with reference to the drawings. In the present disclosure, the drawings used in description of each example embodiment are associated with one or more example embodiments. Elements included in each drawing may apply to one or more example embodiments. The example embodiments described below have technically preferable limitations for carrying out the present disclosure, but the scope of the disclosure is not limited to the following. All the drawings used in the following description of the example embodiments indicate similar parts with the same reference signs unless otherwise specified. In the following example embodiments, repeated description of similar configurations and operations may be omitted in some cases. The directions of the arrows in the drawings indicate examples of flows of signals, data, and the like and do not limit the flows of signals, data, and the like.

### First Example Embodiment

First, an example of a gait measurement system in a first example embodiment will be described with reference to the drawings. The gait measurement system in the present example embodiment measures sensor data regarding movement of a foot according to walking of a user. The measured sensor data is used, for example, to evaluate a walking state of the user.

### Configuration

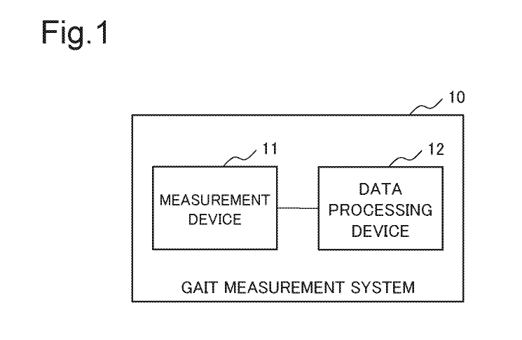

FIG. 1 is a block diagram illustrating a configuration of the gait measurement system in the present disclosure. A gait measurement system 10 includes a measurement device 11 and a data processing device 12. For example, the measurement device 11 is installed on footwear of the user who is a physical condition estimation target. For example, a function of the data processing device 12 is added to the measurement device 11 installed on the footwear of the user who is the physical condition estimation target. For example, a function of the data processing device 12 is installed on a mobile terminal used by the user. For example, a function of the data processing device 12 may be implemented in a server or a cloud accessible from the mobile terminal used by the user. Hereinafter, configurations of the measurement device 11 and the data processing device 12 will be individually described.

### Measurement Device

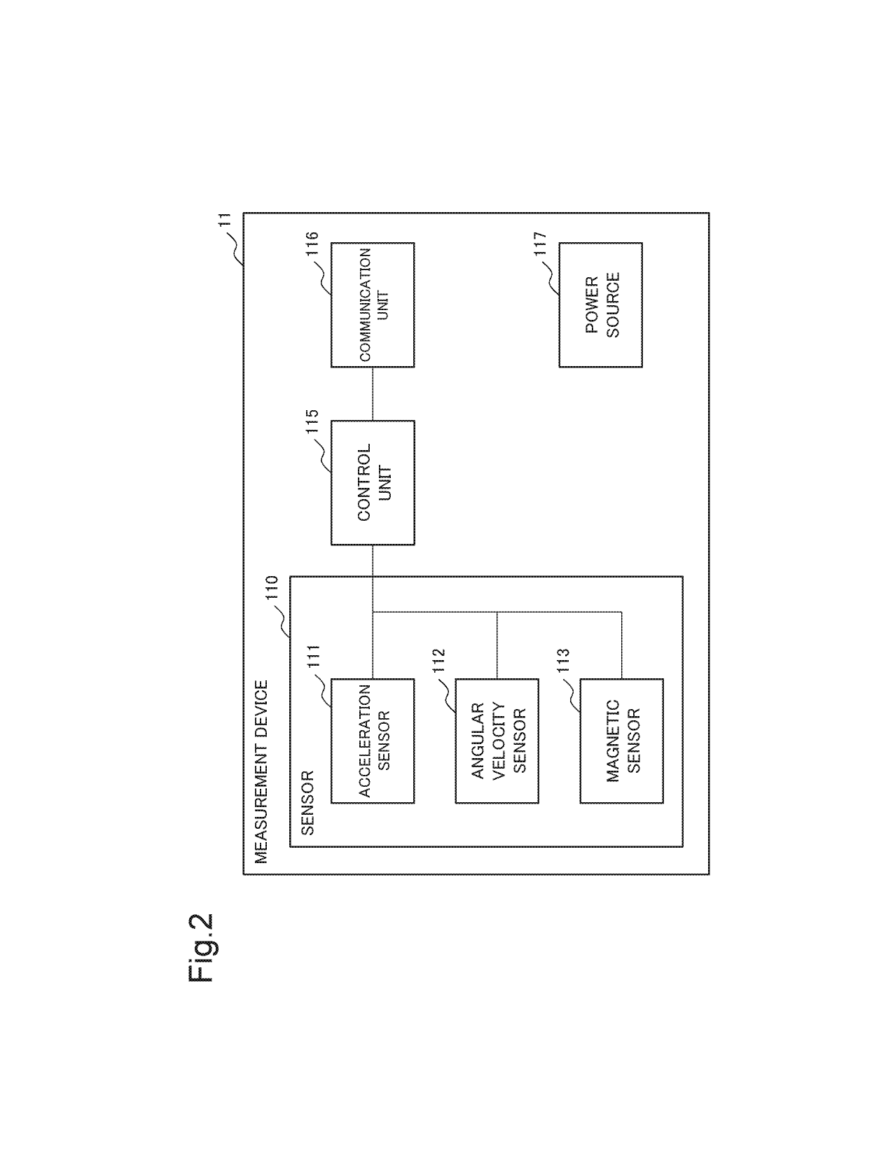

FIG. 2 is a block diagram illustrating an example of the configuration of the measurement device in the present disclosure. The measurement device 11 includes a sensor 110, a control unit 115, a communication unit 116, and a power source 117. The sensor 110 includes an acceleration sensor 111, an angular velocity sensor 112, and a magnetic sensor 113. The sensor 110 may include a sensor other than the acceleration sensor 111, the angular velocity sensor 112, and the magnetic sensor 113. Description of the sensor other than the acceleration sensor 111, the angular velocity sensor 112, and the magnetic sensor 113 that can be included in the sensor 110, will be omitted.

The acceleration sensor 111 is a sensor that measures accelerations (also referred to as spatial accelerations) in three axial directions. The acceleration sensor 111 measures acceleration as a physical quantity related to the movement of the foot. The acceleration sensor 111 outputs the measured acceleration to the control unit 115. For example, a sensor of a piezoelectric type, a piezoresistive type, a capacitance type, or the like can be used as the acceleration sensor 111. The sensor used as the acceleration sensor 111 is not limited as long as the sensor can measure acceleration.

The angular velocity sensor 112 is a sensor that measures an angular velocity (also referred to as a spatial angular velocity) around three axes. The angular velocity sensor 112 measures an angular velocity as a physical quantity related to the movement of the foot. The angular velocity sensor 112 outputs the measured angular velocity to the control unit 115. For example, a sensor of a vibration type, a capacitance type, or the like can be used as the angular velocity sensor 112. The sensor used as the angular velocity sensor 112 is not limited as long as the sensor can measure the angular velocity.

The magnetic sensor 113 is a sensor that measures magnetism (also referred to as magnetic data) in three axial directions. The magnetic sensor 113 outputs the measured magnetic data to the control unit 115. The magnetic data output to the control unit 115 is synchronized with the acceleration and the angular velocity. In the present example embodiment, a change in direction during walking of the user is detected by detecting geomagnetism in the three axial directions. For example, the magnetic sensor 113 is configured to detect a change in a traveling direction of the user. A rapid direction change or an abnormal movement during walking can be detected by a temporal change in magnetic data output from the magnetic sensor 113. For example, in a case where a change in an azimuth angle between pieces of magnetic data that are temporally continuous exceeds a threshold value (for example, 90 degrees), the pieces of magnetic data measured during this period become rejection targets. For example, the magnetic sensor 113 detects magnetism in the same axial direction as the acceleration sensor 111 and the angular velocity sensor 112. As long as a change in the traveling direction of the user can be detected, the axial direction of magnetism detected by the magnetic sensor 113 may be different from those of the acceleration sensor 111 and the angular velocity sensor 112.

For example, a Hall element can be used as the magnetic sensor 113. For example, a Hall effect type semiconductor magnetic sensor has characteristics of a small size, high sensitivity, and low power consumption. The Hall effect type semiconductor magnetic sensor has a measurement range of about ±200 microtesla, sufficiently covers the strength of geomagnetism, and can detect a minute change in direction with a resolution of about 0.1 microtesla. For example, when a sampling rate of the magnetic sensor 113 is set to 50 hertz or more, it is possible to accurately detect a sudden change in direction. For example, as the magnetic sensor 113, a magnetoresistive element such as a semiconductor magnetoresistive element, an anisotropic magnetoresistive element, a giant magnetoresistive element, or a tunnel magnetoresistive element may be used.

For example, the sensor 110 is implemented by adding the magnetic sensor 113 to an inertial measurement device that measures acceleration and an angular velocity. An example of the inertial measurement device is an inertial measurement unit (IMU). The IMU includes an acceleration sensor 111 that measures acceleration in three axial directions and an angular velocity sensor 112 that measures the angular velocity around the three axes. The sensor 110 may be implemented by an inertial measurement device such as a vertical gyro (VG) or an attitude heading reference system (AHRS). The sensor 110 may be implemented by a device other than the inertial measurement device as long as the sensor 110 can measure a physical quantity related to the movement of the foot. For example, the magnetic sensor 113 may be configured as hardware different from the sensor 110 including the acceleration sensor 111 and the angular velocity sensor 112.

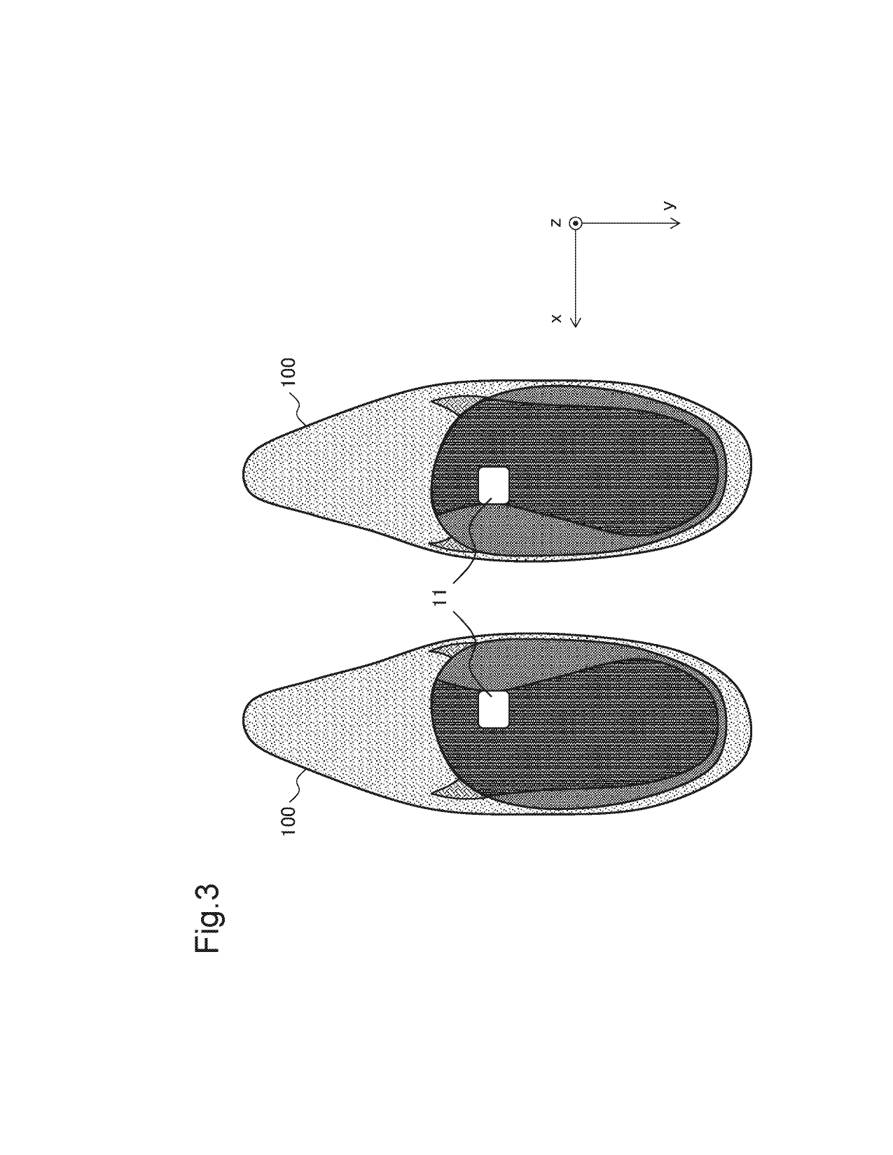

FIG. 3 is a conceptual diagram illustrating an example in which the measurement device in the present disclosure is disposed in a shoe. In the example of FIG. 3, the measurement device 11 is installed at a position relevant to the back side of the arch of a foot. For example, the measurement device 11 is disposed in an insole inserted into a shoe 100. For example, the measurement device 11 may be disposed on the bottom surface of the shoe 100. For example, the measurement device 11 may be embedded in the main body of the shoe 100. The measurement device 11 may be detachable from the shoe 100 or may not be detachable from the shoe 100. The measurement device 11 may be installed at a position other than the back side of the arch of the foot as long as the sensor data related to the movement of the foot can be measured. The measurement device 11 may be installed on a sock worn by the user or a decorative article such as an anklet worn by the user. The measurement device 11 may be directly attached to the foot or may be embedded in the foot. As long as the data from which the physical condition can be estimated can be measured, the measurement device 11 may be disposed in one shoe 100. In a case where data of both feet is required, it is preferable that the measurement devices 11 are arranged in both shoes 100.

In the example of FIG. 3, a local coordinate system including an x axis in the left-right direction, a y axis in the front-rear direction, and a z axis in the upward-downward direction is set with reference to the measurement device 11 (sensor 110). In the present disclosure, the left side of the x axis is positive, the front side of the y axis is positive, and the upper side of the z axis is positive. The positive and negative directions of the x axis, the y axis, and the z axis can be freely set. FIG. 3 illustrates an example in which the same coordinate system is set for a left foot and a right foot. For example, in a case where the sensors 110 produced with the same specifications are disposed in left and right shoes 100, the upward-downward directions (Z-axis direction) of the sensors 110 disposed in the left and right shoes 100 are the same. In this case, the three axes of the local coordinate system set in the sensor data derived from the left foot and the three axes of the local coordinate system set in the sensor data derived from the right foot are the same. Different coordinate systems may be set for the left foot and the right foot.

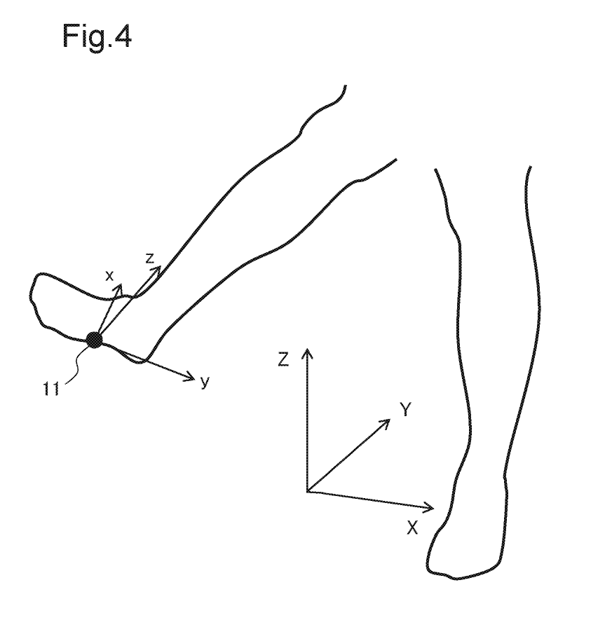

FIG. 4 is a conceptual diagram for describing a local coordinate system and a world coordinate system in the present disclosure. The world coordinate system (X axis, Y axis, Z axis) is set with respect to the ground. The local coordinate system (x axis, y axis, z axis) is set for the measurement device. In the world coordinate system (X axis, Y axis, Z axis), a lateral direction of the user is set to an X-axis direction, a front-rear direction of the user is set to a Y-axis direction, and a vertical direction is set to the Z-axis direction in a state where the user facing the traveling direction is upright. The example of FIG. 4 conceptually illustrates the relationship between the local coordinate system (x axis, y axis, z axis) and the world coordinate system (X axis, Y axis, Z axis), and does not accurately illustrate the relationship between the local coordinate system and the world coordinate system that varies depending on walking of the user.

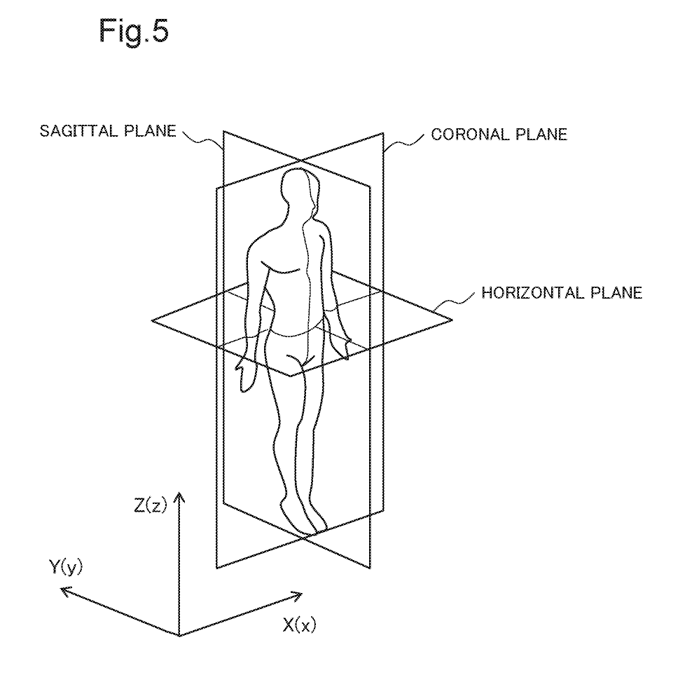

FIG. 5 is a conceptual diagram for describing a human body plane set for a human body. In the present example embodiment, a sagittal plane, a coronal plane, and a horizontal plane are defined. The sagittal plane is a human body plane that divides the body into right and left parts. The coronal plane is a human body plane that divides the body into front and rear parts. The horizontal plane is a human body plane that horizontally divides the body. As illustrated in FIG. 5, it is assumed that the world coordinate system and the local coordinate system coincide with each other in a state in which the center line of the foot is directed upright in the traveling direction. FIG. 5 illustrates an example in which different coordinate systems are set for the left foot and the right foot. In the present example embodiment, rotation in the sagittal plane with the X axis (x axis) as a rotation axis is defined as a roll, rotation in the coronal plane with the Y axis (y axis) as a rotation axis is defined as a pitch, and rotation of the horizontal plane with the Z axis (z axis) as a rotation axis is defined as a yaw. A rotation angle in the sagittal plane with the X axis (x axis) as a rotation axis is defined as a roll angle, a rotation angle in the coronal plane with the Y axis (y axis) as a rotation axis is defined as a pitch angle, and a rotation angle of the horizontal plane with the Z axis (z axis) as a rotation axis is defined as a yaw angle.

The control unit 115 causes the acceleration sensor 111 and the angular velocity sensor 112 to measure sensor data. For example, the control unit 115 causes the acceleration sensor 111 and the angular velocity sensor 112 to start measurement in response to a measurement start signal transmitted from the data processing device 12. For example, the control unit 115 may cause the acceleration sensor 111 and the angular velocity sensor 112 to start measurement at a timing at which walking by the user is detected. For example, after the heights of both legs/feet in the vertical direction are the same over a predetermined period set in advance, the control unit 115 starts the measurement of the sensor data from a time point at which movement of one of the right and left feet in the traveling direction is detected as a starting point. The control unit 115 may be configured to start the measurement of the sensor data at a predetermined timing set in advance. The control unit 115 may be configured to cause the magnetic sensor 113 to measure magnetic data. Description of control of the magnetic sensor 113 by the control unit 115 will be omitted.

The control unit 115 acquires accelerations in three axial directions from the acceleration sensor 111. The control unit 115 acquires the angular velocity around three axes from the angular velocity sensor 112. Furthermore, the control unit 115 acquires magnetic data in three axial directions from the magnetic sensor 113. For example, the control unit 115 performs analog-to-digital conversion (AD conversion) on the acquired physical quantities (analog data) such as the angular velocity and acceleration, and on the magnetic data. The physical quantities (analog data) measured by the acceleration sensor 111 and the angular velocity sensor 112 may be converted into digital data in each of the acceleration sensor 111 and the angular velocity sensor 112. Similarly, the magnetic data measured by the magnetic sensor 113 may be converted into digital data in the magnetic sensor 113. For example, the control unit 115 is provided with an AD conversion circuit that converts analog data into digital data. The control unit 115 outputs the converted digital data (also referred to as sensor data) to the communication unit 116. The control unit 115 synchronizes acceleration data, angular velocity data, and magnetic data included in the sensor data with each other. The control unit 115 may temporarily store the sensor data in the storage unit (not illustrated).

The sensor data includes acceleration data, angular velocity data, and magnetic data. The acceleration data, the angular velocity data, and the magnetic data are converted into digital data. The acceleration data includes acceleration vectors in three axial directions. The angular velocity data includes angular velocity vectors around three axes. The magnetic data includes magnetic vectors in three axial directions. The acceleration data, the angular velocity data, and the magnetic data are associated with acquisition times of these pieces of data. The control unit 115 may be configured to add corrections such as mounting error correction, temperature correction, and linearity correction to the acceleration data, the angular velocity data, and the magnetic data.

For example, the control unit 115 is implemented by a microcomputer or a microcontroller that performs overall control and data processing of the measurement device 11. For example, the control unit 115 includes a central processing unit (CPU), a random access memory (RAM), a read only memory (ROM), a flash memory, and the like.

The communication unit 116 acquires sensor data from the control unit 115. The communication unit 116 transmits the acquired sensor data to the data processing device 12. The sensor data transmitted from the communication unit 116 is received by the data processing device 12. A transmission timing of the sensor data is not particularly limited. For example, the communication unit 116 transmits sensor data at a transmission timing set in advance. For example, the communication unit 116 transmits the sensor data in real time in response to the measurement of the sensor data. For example, the communication unit 116 may store sensor data measured during a predetermined period and collectively transmit the stored sensor data at a preset timing. For example, the communication unit 116 may be configured to receive the measurement start signal from the data processing device 12. In this case, the communication unit 116 outputs the received measurement start signal to the control unit 115.

For example, the communication unit 116 transmits sensor data to the data processing device 12 via wireless communication. For example, the communication unit 116 transmits sensor data to the data processing device 12 via a wireless communication function (not illustrated) conforming to a standard such as Bluetooth (registered trademark) or WiFi (registered trademark). The communication function of the communication unit 116 may conform to a standard other than Bluetooth (registered trademark) or WiFi (registered trademark). The communication unit 116 may transmit the sensor data to the data processing device 12 via a cable in a wired manner.

The power source 117 is a battery that supplies power for the measurement device 11 to operate. For example, the power source 117 is implemented by a thin battery such as a coin type or a button type. For example, the power source 117 is implemented by a primary battery such as a lithium primary battery, a silver oxide battery, an alkaline button battery, or an air zinc battery. In the case of being implemented by the primary battery, the power source 117 is preferably implemented by a long-life battery. The power source 117 may be implemented by a rechargeable secondary battery. In the case of being implemented by the secondary battery, the power source 117 may be a battery that can be charged in a wired manner or may be a battery that can be charged wirelessly. When the power source 117 can wirelessly supply power, the wireless power supply device may be disposed at a place where footwear is placed, such as an entrance or a footwear box. When the footwear on which the measurement device 11 is mounted is stacked on the wireless power supply device, the measurement device 11 can be charged appropriately when not in use.

### Data Processing Device

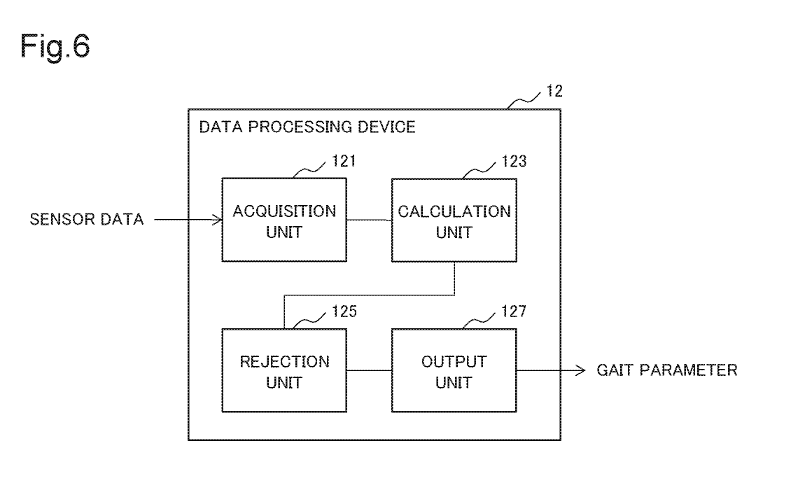

FIG. 6 is a block diagram illustrating an example of a configuration of the data processing device in the present disclosure. The data processing device 12 includes an acquisition unit 121, a calculation unit 123, a rejection unit 125, and an output unit 127.

The acquisition unit 121 acquires time-series data of sensor data from the measurement device 11. The acquisition unit 121 receives the time-series data of the sensor data from the measurement device 11 via wireless communication. For example, the acquisition unit 121 receives the time-series data of the sensor data from the measurement device 11 via a wireless communication function (not illustrated) conforming to a standard such as Bluetooth (registered trademark) or WiFi (registered trademark). The communication function of the acquisition unit 121 may conform to a standard other than Bluetooth (registered trademark) or WiFi (registered trademark) as long as the communication function can communicate with the measurement device 11. The acquisition unit 121 may receive the time-series data of the sensor data from the measurement device 11 via a wire such as a cable.

The calculation unit 123 extracts sensor data for a specific section from the acquired time-series data of the sensor data. The calculation unit 123 extracts sensor data for one gait cycle from the time-series data of the sensor data. For example, the calculation unit 123 may be configured to extract sensor data for several gait cycles from the time-series data of the sensor data. The calculation unit 123 extracts an end point of the gait cycle from the time-series data of the sensor data. The end point is relevant to a timing at which the same gait event is detected. A section between consecutive end points is relevant to one gait cycle. Among two consecutive end points, an end point preceding in time series is set as a start point of one gait cycle. Among the two consecutive end points, an end point later in time series is set as an end point of one gait cycle. The calculation unit 123 extracts time-series data of sensor data between two consecutive end points as a gait waveform for one gait cycle. The calculation unit 123 may normalize the extracted gait waveform. For example, the calculation unit 123 normalizes the time of the extracted gait waveform for one gait cycle to a gait cycle of 0 to 100% (percent) (first normalization). A section such as 1% or 10% included in the gait cycle of 0 to 100% is also referred to as a gait phase. For example, the calculation unit 123 normalizes the gait waveform subjected to the first normalization for one gait cycle so that a stance phase becomes 60% and a swing phase becomes 40% (second normalization). When the gait waveform is subjected to the second normalization, it is possible to reduce the shift of the gait phase from which a feature quantity used to estimate the walking state is extracted.

For example, the end point of the gait cycle is set to a mid-stance period or a timing of heel strike. The timing at the midpoint between the timing at which the roll angle is minimum and the timing at which the roll angle is maximum is relevant to a mid-stance period. In this case, a section between consecutive mid-stance periods is relevant to one gait cycle. The timing of the heel strike is the timing of a local minimum peak immediately after a local maximum peak appearing in the time-series data of the acceleration in the traveling direction (acceleration in a Y direction). The local maximum peak serving as a mark of a heel strike timing is relevant to the maximum peak of the gait waveform for one gait cycle. A section between consecutive mid-stance periods or heel strikes is relevant to one gait cycle. In this case, a section between consecutive heel strikes is relevant to one gait cycle. A mid-stance period or the timing of the heel strike is an example, and does not limit the end point of the gait cycle. For example, the end point of the gait cycle may be set to a timing of a gait event such as toe off, opposite foot toe off, heel lift, opposite foot heel strike, foot crossing, and tibial vertical. Description of a method of detecting the timing of the gait event will be omitted.

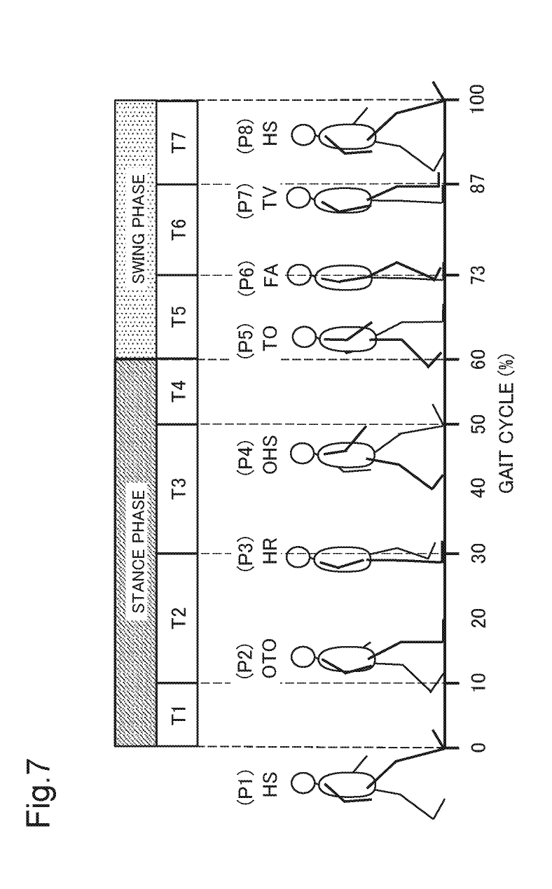

FIG. 7 is a conceptual diagram for describing one gait cycle with the right foot as a reference. One gait cycle based on the left foot is also similar to that of the right foot. The horizontal axis in FIG. 7 indicates one gait cycle of the right foot with a time point at which the heel of the right foot lands on the ground as a starting point and a time point at which the heel of the right foot next lands on the ground as an ending point. The horizontal axis in FIG. 7 is normalized with one gait cycle as 100%. Normalizing one gait cycle by 100% is referred to as first normalization. The one gait cycle of one foot is roughly divided into a stance phase in which at least a part of the back side of the foot is in contact with the ground and a swing phase in which the back side of the foot is separated from the ground. The stance phase is a period in which at least a part of the back side of the foot is in contact with the ground. The stance phase is further subdivided into an initial stance period T1, a mid-stance period T2, a terminal stance period T3, and a pre-swing period T4. The swing phase is a period in which the back side of the foot is away from the ground. The swing phase is further subdivided into an initial swing period T5, a mid-swing period T6, and a terminal swing period T7. The horizontal axis in FIG. 7 is normalized such that the stance phase is 60% and the swing phase is 40%. The normalization of the gait waveform so that the stance phase becomes 60% and the swing phase becomes 40% is referred to as second normalization. The period illustrated in FIG. 7 is an example, and the period constituting one gait cycle, the name of those periods, and the like are not limited.

As illustrated in FIG. 7, a plurality of events occur in walking. In the walking, a plurality of events in the walking are also referred to as gait events. P1 represents an event (heel strike) in which the heel of the right foot touches the ground (HS: Heel Strike). P2 represents an event (opposite toe off) in which the toe of the left foot is separated from the ground in a state where the sole of the right foot is grounded (OTO: Opposite Toe Off). P3 represents an event (heel rise) in which the heel of the right foot lifts in a state where the sole of the right foot is grounded (HR: Heel Rise). P4 is an event (opposite heel strike) in which the heel of the left foot is grounded (OHS: Opposite Heel Strike). P5 represents an event (toe off) in which the toe of the right foot is separated from the ground in a state where the sole of the left foot is grounded (TO: Toe Off). P6 represents an event (foot adjacent) in which the left foot and the right foot cross each other in a state where the sole of the left foot is grounded (FA: Foot Adjacent). P7 represents an event (tibia vertical) in which the tibia of the right foot is approximately perpendicular to the ground in a state where the sole of the left foot is grounded (TV: Tibia Vertical). P8 represents an event (heel strike) in which the heel of the right foot touches the ground (HS: Heel Strike). P8 is relevant to the end point of the gait cycle starting from P1 and is relevant to the start point of the next gait cycle. The gait event illustrated in FIG. 7 is an example, and does not limit the events that occur in the walking or the names of these events.

The timing of the heel strike is the timing of a local minimum peak immediately after a local maximum peak appearing in the time-series data of the acceleration in the traveling direction (acceleration in the Y direction). The local maximum peak serving as a mark of a heel strike timing is relevant to the maximum peak of the gait waveform for one gait cycle. A section between consecutive heel strikes is relevant to one gait cycle. The timing of the toe off is the rising timing of the local maximum peak appearing after the period of the stance phase in which the variation does not appear in the time-series data of the acceleration in the traveling direction (acceleration in the Y direction). The timing at the midpoint between the timing at which the roll angle is minimum and the timing at which the roll angle is maximum is relevant to a mid-stance period.

For example, the calculation unit 123 extracts the gait waveform for one gait cycle by using the acceleration in the traveling direction (acceleration in the Y direction). In this case, regarding acceleration, an angular velocity, and an angle other than the acceleration in the traveling direction (acceleration in the Y direction), the calculation unit 123 extracts the gait waveform for one gait cycle with a gait cycle of the acceleration in the traveling direction (acceleration in the Y direction). The calculation unit 123 extracts a gait waveform related to acceleration in three axial directions, a gait waveform related to an angular velocity around three axes, and a gait waveform related to an angle around the three axes. The calculation unit 123 may generate time-series data of the angle around the three axes by integrating time-series data of the angular velocity around the three axes. The calculation unit 123 normalizes the extracted gait waveform for one gait cycle.

The calculation unit 123 may extract the gait waveform for one gait cycle by using the acceleration and the angular velocity other than the acceleration in the traveling direction (acceleration in the Y direction). For example, the calculation unit 123 detects the heel strike and the toe off from the time-series data of acceleration in the vertical direction (acceleration in the Z-direction). The timing of the heel strike is a timing of a steep local minimum peak appearing in the time-series data of the acceleration in the vertical direction (acceleration in the Z direction). At the timing of the steep local minimum peak, the value of the acceleration in the vertical direction (acceleration in the Z direction) becomes substantially zero. The local minimum peak serving as a mark of the timing of the heel strike is relevant to the minimum peak of the gait waveform for one gait cycle. A section between consecutive heel strikes is one gait cycle. The timing of the toe off point is a timing of an inflection point in the middle of gradually increasing after the time-series data of the acceleration in the vertical direction (acceleration in the Z direction) passes through a section with a small variation after the local maximum peak immediately after the heel strike. The calculation unit 123 may extract the gait waveform for one gait cycle by using both the acceleration in the traveling direction (acceleration in the Y direction) and the acceleration in the vertical direction (acceleration in the Z direction). The calculation unit 123 may extract the gait waveform for one gait cycle by using the acceleration, the angular velocity, the angle, and the like other than the acceleration in the traveling direction (acceleration in the Y direction) and the acceleration in the vertical direction (acceleration in the Z direction).

The calculation unit 123 calculates a gait parameter by using the extracted sensor data for a specific period. The gait parameter is an index quantitatively indicating a walking motion. For example, the calculation unit 123 calculates the gait parameter by using the normalized gait waveform. For example, the calculation unit 123 calculates a posture angle of the measurement device 11 (sensor 110) by using a Madgwick filter. For example, the calculation unit 123 calculates the gait parameter related to a distance, a height, an angle, a speed, a time, a center of pressure exclusion index (CPEI), a frailty level, and the like. The gait parameter is used to estimate a physical condition, physical ability, and the like. The gait parameter calculated by the calculation unit 123 is not particularly limited.

The gait parameter related to the distance and the height includes a stride length, an outward turning distance, a foot raising height, foot clearance (FTC), and minimum toe clearance (MTC). The stride length indicates a distance between a front foot and a rear foot during walking. The outward turning distance indicates the maximum value of the distance at which the foot is separated outward with respect to the traveling direction in the swing phase. The foot raising height indicates the maximum value of the distance between the measurement device 11 (sensor 110) and the ground in the swing phase. The FTC indicates the maximum value of the distance between the heel and the ground in the swing phase. The MTC indicates the minimum value of the distance between the toe and the ground in the swing phase.

The gait parameter related to the angle includes a grounding angle, a ground separation angle, a toe direction, a roll angle of heel strike, a roll angle of toe off, a swing peak angular velocity, and a hallux angle. The grounding angle indicates a maximum value of an angle formed by the sole surface and the ground at the time of heel strike. The ground separation angle indicates an angle formed between the sole surface and the ground in the swing phase. The toe direction indicates an average value of the toe directions with respect to the traveling direction in the swing phase. The roll angle of the heel strike is an angle formed between the ankle and the ground at the time of the heel strike when viewed from the rear viewing seat. The roll angle of the toe off is an angle formed between the ankle and the ground at the time of kicking as viewed from the rear viewing seat. The swing peak angular velocity is the angular velocity in the ankle joint dorsiflexion direction in a section from immediately after kicking until the toe comes into closest contact with the ground. The hallux angle indicates an angle at which the big toe of the foot is inclined toward the second toe. Specifically, the hallux angle is an angle formed by the center line of the first metatarsal and the center line of the first proximal phalanx.

The gait parameter related to the speed includes a walking speed, cadence, and a maximum speed in swing. The walking speed indicates a speed in the gait. The cadence indicates the number of steps per minute. The maximum speed in swing indicates a speed at which the user swings out the leg in the swing phase.

The gait parameter related to the time includes a standing time, a load time, a plantar grounding time, a kicking time, a swing time, and a double support time (DST). The standing time indicates a time during which the foot is grounded during walking. The standing time is a sum of the load time, the plantar grounding time, and the kicking time. The load time is a time from when the heel is grounded to when the toe is grounded in the stance phase. The plantar grounding time is a time during which the entire plantar surface is grounded and the plantar surface and the ground are horizontal in the stance phase. The kicking time is a time until the toe kicks the ground from the state of the sole grounding in the stance phase. The swing time indicates a time during which the foot is separated from the ground during walking. The DST is divided into DST1 and DST2. DST1 indicates a time during which the foot on which the measurement device 11 (sensor 110) is mounted is in front of the opposite foot in a period in which both feet are simultaneously grounded. DST2 indicates a time during which the foot on which the measurement device 11 (sensor 110) is mounted is behind the opposite foot in a period in which both feet are simultaneously grounded.

The CPEI indicates an estimated value of the expansion ratio of the movement of the center of foot pressure applied to the ground during the stance phase. The frailty level is an estimated value of frailty detected from the walking state. For example, the calculation unit 123 estimates the possibility of frailty as the frailty level. In a case where there is no possibility of frailty, the calculation unit 123 outputs a determination result indicating that there is no frailty. In a case where there is a possibility of frailty, the calculation unit 123 outputs a determination result indicating that there is a possibility of frailty. In a case where the possibility of frailty is high, the calculation unit 123 outputs a determination result indicating that the possibility of frailty is high.

The calculation unit 123 may be configured to extract a feature quantity used to calculate and estimate the gait parameter from the gait waveform. For example, the calculation unit 123 extracts the feature quantity for each gait phase cluster in accordance with a condition set in advance. The gait phase cluster is a cluster in which temporally continuous gait phases are integrated. The gait phase cluster includes at least one gait phase. The gait phase cluster can also include a single gait phase. The calculation unit 123 may extract a physical ability feature quantity used to estimate the physical ability. For example, the physical ability feature quantity is used to estimate physical ability such as grip strength (total muscle strength of the whole body), dynamic balance, lower limb muscle strength, movement ability, and static balance.

The rejection unit 125 calculates an azimuth angle by using magnetic data included in sensor data. The rejection unit 125 detects a variation in azimuth angle. The rejection unit 125 calculates a difference in the azimuth angle between pieces of the magnetic data as comparison targets, as the variation in the azimuth angle. A method of selecting the magnetic data as the comparison target is not particularly limited. For example, the magnetic data as the comparison target is temporally continuous magnetic data. For example, the magnetic data as the comparison target is magnetic data measured at predetermined time intervals.

Sensor data measured in a time zone in which the variation in the azimuth angle does not exceed a threshold value is normal data. Therefore, the rejection unit 125 outputs the gait parameter itself calculated by using the sensor data (normal data) measured in the time zone in which the variation in the azimuth angle does not exceed the threshold value. Sensor data measured in the time zone in which the variation in the azimuth angle is equal to or greater than the threshold value is abnormality data. Therefore, the rejection unit 125 rejects the gait parameter calculated by using the sensor data (abnormality data) measured in the time zone in which the variation in the azimuth angle is equal to or greater than the threshold value. For example, the threshold value is set to 90 degrees. In this case, the rejection unit 125 rejects the gait parameter calculated by using the sensor data (abnormality data) measured in the time zone in which the variation in the azimuth angle is 90 degrees or more. The threshold value set for the variation in the azimuth angle can be freely set.

For example, the rejection unit 125 rejects the sensor data according to a variation in the gait parameter that is susceptible to a posture angle, such as a foot raising height, a plantarflexion angle, a dorsiflexion angle, FTC, and MTC. For example, in a case where any one of the gait parameters varies 1.5 times or more from the previous sampling timing, the rejection unit 125 rejects sensor data as a calculation source of these gait parameters as abnormality data. In normal walking, the plantarflexion angle is larger than the dorsiflexion angle. Therefore, in a case where the plantarflexion angle is smaller than the dorsiflexion angle, sensor data as the calculation source of the dorsiflexion angle and the plantarflexion angle becomes a rejection target as the abnormality data. For example, the rejection unit 125 may be configured to verify whether the azimuth angle shows a variation equal to or greater than a threshold value for sensor data as the calculation source of the gait parameter that has varied by a reference value or more, which is set in advance (for example, 1.5 times or more). With this configuration, since the abnormality data can be detected in two stages by the variation in the gait parameter and the azimuth angle, it is possible to more precisely reject the abnormality data.

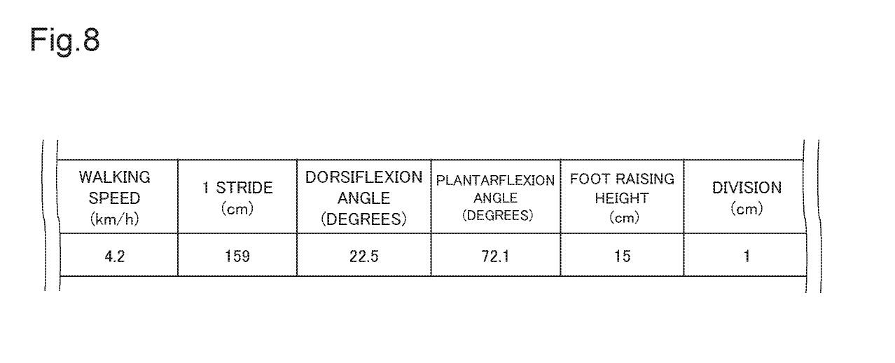

FIG. 8 illustrates an example of a table in which values of gait parameters calculated by the data processing device included in the gait measurement system in the present disclosure are collected. The table of FIG. 8 shows an example of gait parameters calculated by using sensor data measured in a 10 m (meter) walking test performed indoors. FIG. 8 illustrates gait parameters calculated by using normal data. The walking speed is 4.2 km/h (kilometers per hour). 1 stride is 159 cm (centimeters). The dorsiflexion angle is 22.5 degrees. The plantarflexion angle is 72.1 degrees. The foot raising height is 15 cm. The division is 1 cm. In normal walking, the plantarflexion angle is larger than the dorsiflexion angle.

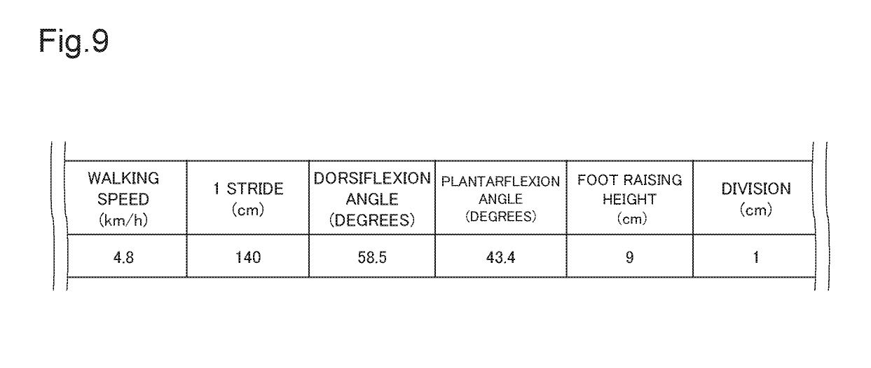

FIG. 9 illustrates another example of the table in which the values of the gait parameters calculated by the data processing device included in the gait measurement system in the present disclosure are collected. The table of FIG. 9 shows an example of gait parameters calculated by using sensor data measured in a 10 m (meter) walking test performed indoors. FIG. 9 illustrates gait parameters calculated by using abnormality data. The walking speed is 4.8 km/h (kilometers per hour). 1 stride is 140 cm (centimeters). The dorsiflexion angle is 58.5 degrees. The plantarflexion angle is 43.4 degrees. The foot raising height is 9 cm. The division is 1 cm. In the example of FIG. 9, since the plantarflexion angle is smaller than the dorsiflexion angle, it is estimated that the abnormality data is measured due to a sudden direction change or the like. When the abnormality data is used for calculation of the gait parameter, it is not possible to accurately estimate the physical condition of the user. According to the method in the present example embodiment, by rejecting the abnormality data according to the variation in the azimuth angle, the abnormality data in which the plantarflexion angle is smaller than the dorsiflexion angle is deleted. Therefore, according to the method in the present example embodiment, it is possible to accurately estimate the physical condition of the user by using the normal data that does not include the abnormality data.

The output unit 127 outputs the gait parameter calculated by using the normal data. For example, the output unit 127 outputs the gait parameter to a mobile terminal used by the user. For example, the output unit 127 outputs the gait parameter to a terminal device or a server via the mobile terminal used by the user. For example, the output unit 127 may be configured to output the gait parameter to an external system or the like.

For example, the data processing device 12 is constructed in a cloud or a server connected to a mobile terminal used by a user via a communication network. The mobile terminal is a portable communication device. For example, the mobile terminal is a portable communication device having a communication function, such as a smartphone, a smart watch, or a mobile phone. For example, the data processing device 12 is connected to a mobile terminal via wireless communication. For example, the data processing device 12 is connected to a mobile terminal via a wireless communication device (not illustrated) conforming to a standard such as Bluetooth (registered trademark) or WiFi (registered trademark). The wireless communication device may conform to a standard other than Bluetooth (registered trademark) or WiFi (registered trademark). The gait parameter may be used by an application installed on the mobile terminal. For example, the mobile terminal executes processing using the gait parameter by an application installed on the mobile terminal.

### Operation

Next, an example of an operation of the data processing device in the present disclosure will be described with reference to the drawings. The following description of operations (data processing methods) is schematic. Details of the following operation (data processing method) are as described in the above-described configuration.

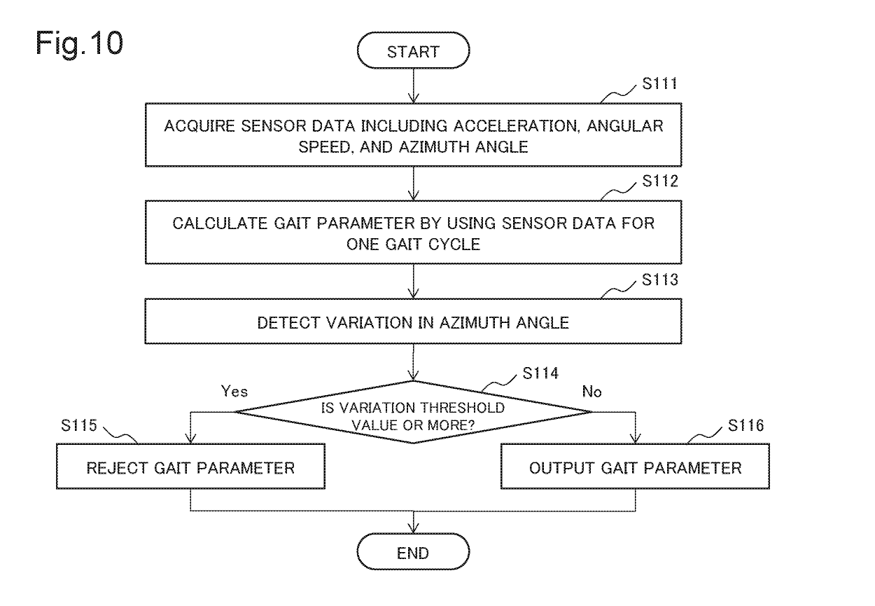

FIG. 10 is a flowchart illustrating an example of the operation of the data processing device in the present disclosure. In the description of the processing as per the flowchart in FIG. 10, a component of the data processing device 12 is assumed as an operating subject. The operating subject of the processing (data processing) as per the flowchart of FIG. 10 may be the data processing device 12. For example, the data processing is achieved by a processor executing a program stored in a memory mounted in a computer (not illustrated) in which the functions of the data processing device 12 are implemented.

In FIG. 10, first, the acquisition unit 121 acquires sensor data including acceleration, an angular velocity, and an azimuth angle (Step S111).

The calculation unit 123 calculates the gait parameter by using the sensor data for one gait cycle (Step S112).

The rejection unit 125 detects a variation in azimuth angle (Step S113).

In a case where the variation in the azimuth angle is equal to or larger than the threshold value (Yes in Step S114), the rejection unit 125 rejects the gait parameter (Step S115).

On the other hand, in a case where the variation in the azimuth angle does not exceed the threshold value (No in Step S114), the output unit 127 outputs the gait parameter (Step S116).

### Modification Examples

Next, a modification example of the present example embodiment will be described with reference to the drawings. In the present modification example, the variation in the azimuth angle is detected in a previous stage of calculating the gait parameter.

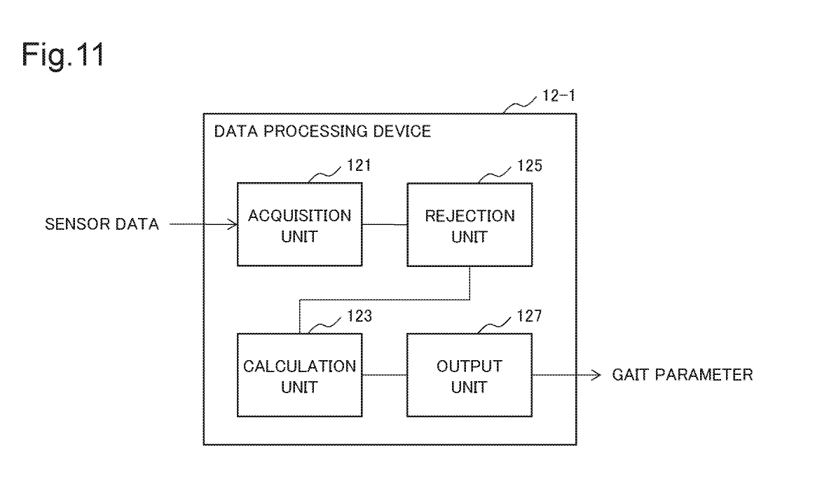

FIG. 11 is a block diagram illustrating an example of a configuration of a data processing device in the present disclosure. A data processing device 12-1 in the present modification example includes an acquisition unit 121, a calculation unit 123, a rejection unit 125, and an output unit 127, similarly to the data processing device 12 (FIG. 6). The data processing device 12-1 is different from the data processing device 12 (FIG. 6) in that the rejection unit 125 is arranged in a preceding stage of the calculation unit 123. Description of processes of the acquisition unit 121, the calculation unit 123, the rejection unit 125, and the output unit 127 will be omitted.

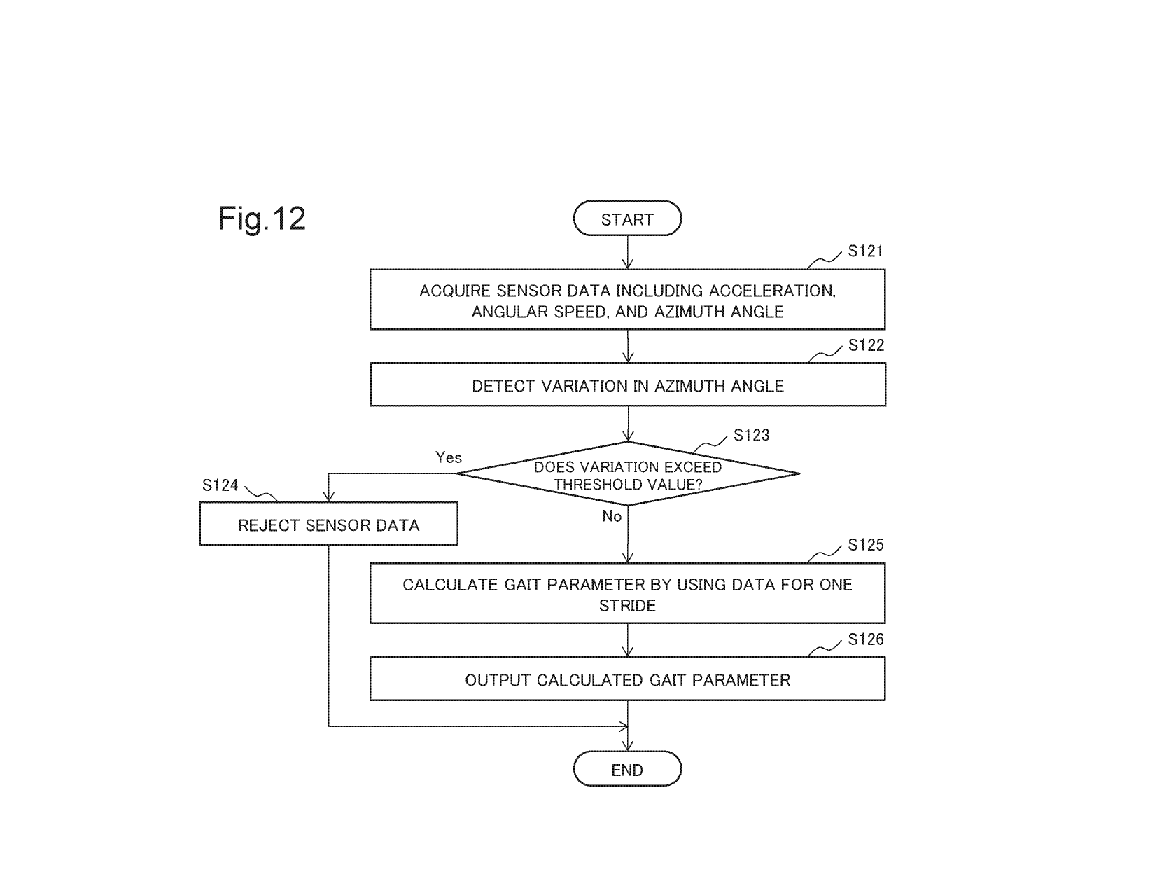

FIG. 12 is a flowchart illustrating an example of an operation of the data processing device in the present disclosure. In the description of the processing as per the flowchart in FIG. 12, a component of the data processing device 12-1 is assumed as an operating subject. The operating subject of the processing (data processing) as per the flowchart of FIG. 12 may be the data processing device 12-1. For example, the data processing is achieved by a processor executing a program stored in a memory mounted in a computer (not illustrated) in which the functions of the data processing device 12-1 are implemented.

In FIG. 12, first, the acquisition unit 121 acquires sensor data including acceleration, an angular velocity, and an azimuth angle (Step S121).

The rejection unit 125 detects a variation in azimuth angle (Step S122).

In a case where the variation in the azimuth angle is equal to or larger than the threshold value (Yes in Step S123), the rejection unit 125 rejects the sensor data (Step S124).

On the other hand, in a case where the variation in the azimuth angle does not exceed the threshold value (No in Step S123), the calculation unit 123 calculates the gait parameter by using the sensor data for one gait cycle (Step S125).

The output unit 127 outputs the gait parameter (Step S126).

In the present modification example, the abnormality data is rejected in the previous stage of calculating the gait parameter. Therefore, according to the present modification example, it is possible to reduce calculation of an incorrect gait parameter using abnormality data. According to the present modification example, since the incorrect gait parameter using the abnormality data is not calculated, the incorrect gait parameter is not erroneously output.

As described above, the gait measurement system in the present example embodiment includes the acquisition unit, the calculation unit, the rejection unit, and the output unit. The acquisition unit acquires sensor data including acceleration data, angular velocity data, and magnetic data measured by the sensor mounted on the footwear of the user. The calculation unit calculates a gait parameter quantitatively indicating the walking motion of the user by using the sensor data from which the abnormality data has been excluded. The rejection unit rejects the abnormality data included in the sensor data according to the variation in the azimuth angle of the sensor calculated by using the magnetic data. The output unit outputs the sensor data from which the abnormality data has been excluded.

In the present example embodiment, the abnormality data included in the sensor data is rejected according to the variation in the azimuth angle of the sensor calculated by using the magnetic data. Therefore, according to the present example embodiment, it is possible to detect a sudden direction change or an abnormal motion during walking and to exclude inappropriate data caused by the sudden direction change or the abnormal motion. Therefore, according to the present example embodiment, it is possible to perform more accurate gait analysis. In the present example embodiment, the gait parameter is calculated by using the sensor data from which the abnormality data has been excluded. Therefore, according to the present example embodiment, it is possible to objectively evaluate the walking state of the user by quantifying and expressing the feature of walking.

For example, when the magnetic data is corrected by using both the acceleration and the angular velocity, it is possible to reduce the influence of a magnetic body around the magnetic sensor and to detect a more accurate change in direction. By using the magnetic data measured by the magnetic sensor, it is possible to accurately detect the change in direction and disturbance of the walking state during walking, and thus, it is possible to perform more precise gait analysis. When the magnetic sensor is used in combination, it is possible to analyze details of the walking variation, which has been difficult only with the acceleration sensor and the angular velocity sensor.

For example, the calculation unit 123 calculates a posture angle of the measurement device (sensor) by using a Madgwick filter. Since the Madgwick filter is a digital filter, convergence is fast in a case where an initial state coincides with a gait state, but it takes nearly 10 seconds until data is stably converged, in a case where a large course change or the like frequently performed in daily walking is performed. Therefore, in the case using the Madgwick filter, the variation in gait data is large, and it is difficult to determine whether the actual sensor data fluctuates. For example, gait parameters such as a foot raising height, a plantarflexion angle, a dorsiflexion angle, FTC, and MTC are susceptible to posture angles. Therefore, the sensor data as the calculation source of the gait parameters that have greatly varied become a rejection target as the abnormality data. For example, in a case where any one of the gait parameters of the foot raising height, the plantarflexion angle, the dorsiflexion angle, FTC, and MTC varies 1.5 times or more from the previous sampling timing, the sensor data as the calculation source of these gait parameters becomes the rejection target as the abnormality data. In addition, in normal walking, the plantarflexion angle is larger than the dorsiflexion angle. Therefore, in a case where the plantarflexion angle is smaller than the dorsiflexion angle, sensor data as the calculation source of the dorsiflexion angle and the plantarflexion angle becomes a rejection target as the abnormality data. In a case where the traveling direction of the user who is walking changes by 90 degrees or more, there is a possibility that it is a timing at which the user approaches a corner or changes the direction. When the sensor data measured at such a timing is used, it is not possible to accurately estimate the physical condition. Therefore, in a case where the azimuth angle measured by the magnetic sensor changes by 90 degrees or more, sensor data measured at the same timing as the azimuth angle becomes the rejection target as the abnormality data.

In the present example embodiment, in addition to the acceleration in a three-dimensional direction (spatial acceleration) and the angular velocity around the three axes (spatial angular velocity), magnetic data in the three axial directions is measured. That is, in the present example embodiment, a compass function by a magnetic sensor is added to the estimation of the posture angle of the measurement device (sensor) by the Madgwick filter using the acceleration and the angular velocity. In the present example embodiment, correctness or incorrectness of the gait parameter measured by using the sensor data is determined by using the azimuth variation obtained from the compass function. According to the present example embodiment, it is possible to detect the direction change and the disturbance of the walking state by using the magnetic data measured by the magnetic sensor and to exclude the abnormality data until the Madgwick filter performs convergence. In the past, the traveling direction and the like have been estimated from the value of division and the like. According to the present example embodiment, the variation in each stride direction becomes clear by using azimuth information, and it is possible to accurately estimate the gait state.

In one aspect of the present example embodiment, the rejection unit calculates the azimuth angle of the sensor by using the magnetic data. The rejection unit rejects sensor data measured at a timing at which the variation in the azimuth angle of the sensor is equal to or greater than the threshold value. In the present aspect, the sensor data measured at the timing at which the variation in the azimuth angle of the sensor is equal to or greater than the threshold value is rejected. According to this correspondence, it is possible to quantitatively determine a large change in direction during walking and to specify abnormality data. Therefore, according to the present aspect, it is possible to appropriately evaluate the stability and continuity of walking.

In one aspect of the present example embodiment, the rejection unit rejects sensor data measured at a timing at which the azimuth angle of the sensor varies by 90 degrees or more. In the present aspect, the sensor data measured at the timing at which the azimuth angle of the sensor varies by 90 degrees or more is rejected. According to the present aspect, it is possible to reliably detect an extreme movement such as a sudden direction change or fall. Therefore, according to the present aspect, it is possible to achieve more reliable gait analysis by using normal data that does not include abnormality data.

In one aspect of the present example embodiment, the rejection unit verifies whether the azimuth angle shows the variation equal to or larger than the threshold value for sensor data as the calculation source of the gait parameter that has varied by a reference or more, which has been set in advance. In the present aspect, the variation of the azimuth angle is not verified for the sensor data as the calculation source of the gait parameter having a small variation. Therefore, by analyzing the abnormal variation of the gait parameter and the variation in the azimuth angle in association with each other, it is possible to more precisely detect the abnormality.

In one aspect of the present example embodiment, the calculation unit calculates the gait parameter by using the sensor data from which the abnormality data has been excluded. According to the present aspect, by calculating the gait parameter by using the normal data including no abnormality data, it is possible to more accurately evaluate the walking state.

In one aspect of the present example embodiment, the calculation unit calculates the posture angle of the sensor by using the Madgwick filter. The calculation unit calculates the gait parameter by using the calculated posture angle of the sensor. According to the present aspect, it is possible to estimate the posture angle of the sensor with high accuracy, and to calculate the gait parameter with high accuracy by using the posture angle. Therefore, according to the present aspect, it is possible to more accurately capture a complicated walking motion and to perform detailed gait analysis. According to the present aspect, it is possible to detect the direction change and the disturbance of the walking state by using the magnetic data measured by the magnetic sensor and to exclude the abnormality data until the Madgwick filter performs convergence.

### Second Example Embodiment

Next, a gait measurement system according to a second example embodiment will be described with reference to the drawings. The gait measurement system in the present example embodiment evaluates the gait of a user by using the gait parameters calculated in the gait measurement system in the first example embodiment.

### Configuration

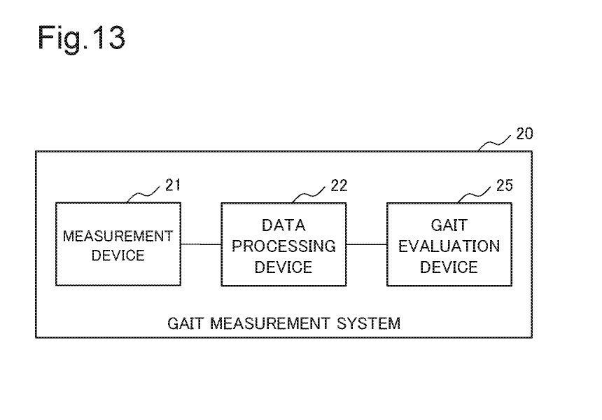

FIG. 13 is a block diagram illustrating a configuration of the gait measurement system in the present disclosure. A gait measurement system 20 in the present example embodiment includes a measurement device 21, a data processing device 22, and a gait evaluation device 25. The measurement device 21 has a configuration similar to the measurement device 11 in the first example embodiment. The data processing device 22 has a configuration similar to the data processing device 12 in the first example embodiment. Therefore, in the present example embodiment, description of the measurement device 21 and the data processing device 22 will be omitted. For example, the function of the gait evaluation device 25 is implemented in a server or a cloud accessible from a mobile terminal used by the user. For example, the function of the gait evaluation device 25 may be installed on the mobile terminal used by the user.

### Gait Evaluation Device

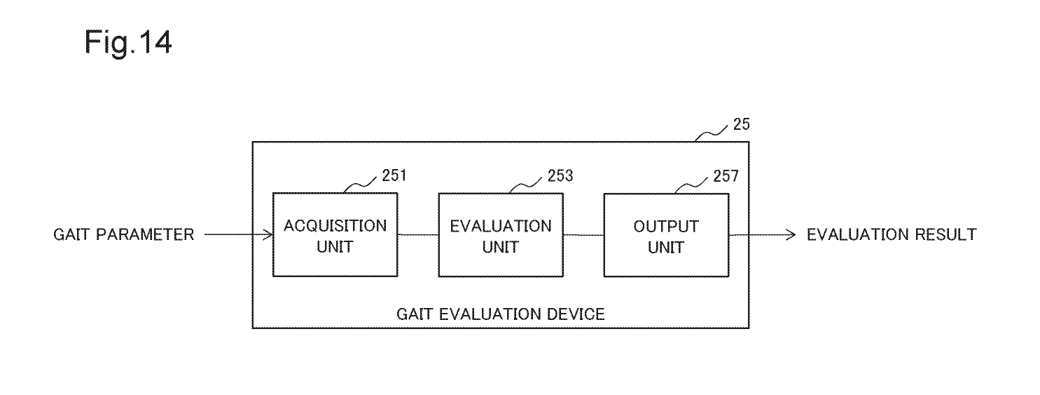

FIG. 14 is a block diagram illustrating an example of a configuration of the gait evaluation device in the present disclosure. The gait evaluation device 25 includes an acquisition unit 251, an evaluation unit 253, and an output unit 257.

The acquisition unit 251 acquires a gait parameter from the data processing device 22. The gait parameter acquired by the acquisition unit 251 is a value calculated by using the normal data.

The evaluation unit 253 evaluates the walking state of the user by analyzing the acquired gait parameter. The evaluation unit 253 may be configured to evaluate not only the walking state but also the physical condition, the health state, and the exercise ability. For example, the evaluation unit 253 quantifies the feature (gait) of walking by using time-series data of the gait parameters by a statistical analysis method or a machine learning algorithm. For example, the evaluation unit 253 evaluates the walking state, the physical condition, the health state, and the exercise ability of the user by using indices such as gait stability, left-right balance, and the degree of fatigue. The evaluation unit 253 may be configured to generate advice according to an evaluation result regarding the walking state of the user.

The greater the change in a traveling direction angle with respect to the walking speed, the higher the gait stability with respect to disturbance. Therefore, in a case where the change in the traveling direction angle with respect to the walking speed is equal to or greater than a reference value, the evaluation unit 253 evaluates that the gait stability is high. For example, the evaluation unit 253 evaluates the walking state of the user based on the temporal fluctuation of the variation value in the traveling direction. The evaluation unit 253 evaluates that the gait stability is low in a case where the sign is reversed in the reference time zone (about several seconds). For example, the evaluation unit 253 evaluates the walking state of the user based on the magnitude of a variation value in the traveling direction. In a case where the variation value is larger than the reference value, the evaluation unit 253 evaluates that the gait stability of the user is low.

For example, the evaluation unit 253 evaluates the walking state and the physical condition of the user according to a difference between the gait parameter of the left foot and the gait parameter of the right foot. Usually, the gait parameter of the weaker foot tends to be smaller than the gait parameter of the non-weakened foot. Therefore, the evaluation unit 253 evaluates that the value of the gait parameter falls below the reference, and the foot having the smaller value is weak. In a case where it is known in advance that one foot of the user is weak, the degree of recovery and the degree of progress can be evaluated according to the temporal change of the difference between the gait parameters of both feet. The references for the degree of recovery and the degree of progress can be freely set. In a case where the difference between the gait parameters of both feet gradually decreases, the evaluation unit 253 evaluates that the symptom of the weaker foot of the user tends to recover. On the other hand, in a case where the difference between the gait parameters of both feet gradually decreases, the evaluation unit 253 evaluates that the symptom of the weaker foot of the user tends to progress.

The output unit 257 outputs the evaluation result by the evaluation unit 253. For example, the output unit 257 outputs the evaluation result to the mobile terminal used by the user. For example, the evaluation result is displayed on a screen of the mobile terminal used by the user. For example, the output unit 257 outputs the evaluation result to a terminal device or a server via the mobile terminal used by the user. For example, the output unit 257 may be configured to output the evaluation result to an external system or the like.

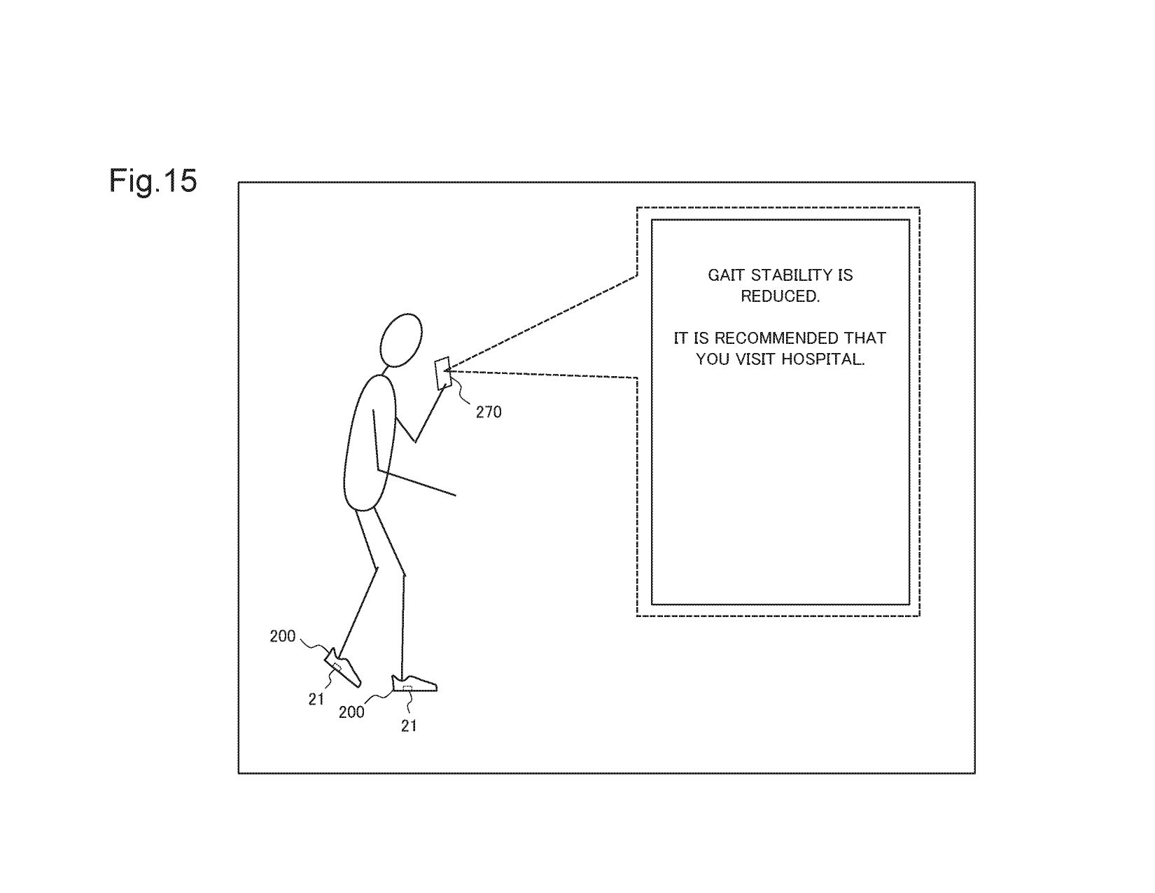

FIG. 15 illustrates a display example of the evaluation result output from the gait evaluation device included in the gait measurement system in the present disclosure. The user is walking while wearing shoes 200 on which the measurement device 21 is mounted. Information regarding the evaluation result by the gait evaluation device 25 is displayed on the screen of a mobile terminal 270 used by the user. In the example of FIG. 15, information according to the evaluation result regarding the walking state of the user such as “gait stability is reduced.” is displayed on the screen of the mobile terminal 270. The user can recognize his/her walking state by checking the evaluation result displayed on the screen of the mobile terminal 270. On the screen of the mobile terminal 270, advice according to the evaluation result regarding the walking state of the user such as “it is recommended that you visit the hospital.” is displayed. The user can check the advice displayed on the screen of the mobile terminal 270 and recognize that it is better to visit the hospital.

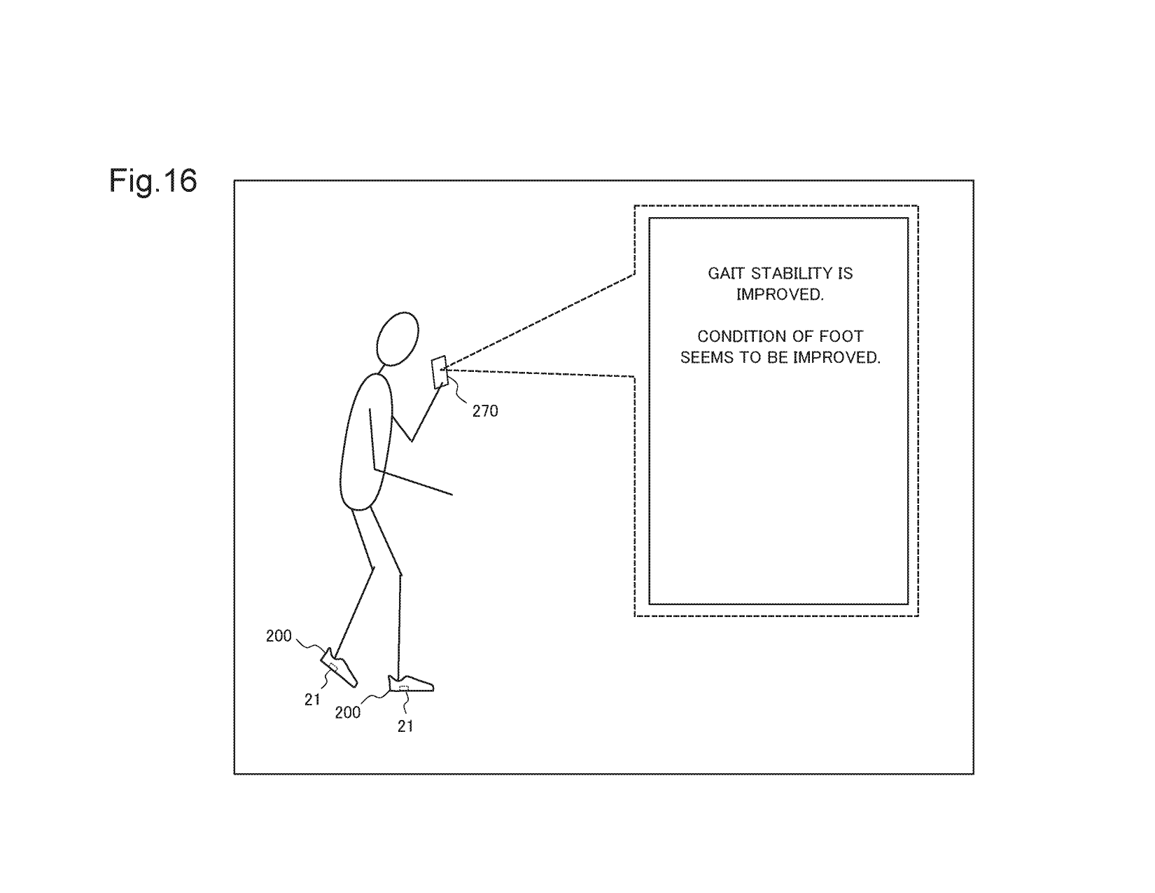

FIG. 16 illustrates another display example of the evaluation result output from the gait evaluation device included in the gait measurement system in the present disclosure. The user is walking while wearing shoes 200 on which the measurement device 21 is mounted. Information regarding the evaluation result by the gait evaluation device 25 is displayed on the screen of a mobile terminal 270 used by the user. In the example of FIG. 16, information according to the evaluation result regarding the walking state of the user such as “gait stability is improved.” is displayed on the screen of the mobile terminal 270. The user can recognize his/her walking state by checking the evaluation result displayed on the screen of the mobile terminal 270. On the screen of the mobile terminal 270, advice according to the evaluation result regarding the walking state of the user such as “the condition of the foot seems to be improved.” is displayed. The advice is optimized according to the physical condition of the user. The advice includes information for prompting the user to make a decision according to the physical condition. The user can check the advice displayed on the screen of the mobile terminal 270 and recognize that his/her weak foot tends to recover.

For example, the gait evaluation device 25 generates advice that is optimized according to the physical condition of the user and assists the user in decision-making, by using a machine learning model (not illustrated). The machine learning model is a model constructed by machine learning to output advice according to the physical condition by inputting the gait parameters. For example, the gait evaluation device 25 optimizes the advice according to the physical condition of the user. The advice includes information for prompting the user to make a decision according to the physical condition. For example, the advice includes information for prompting the user to make a decision on gait improvement. The gait evaluation device 25 outputs the generated advice. The user who has checked the information displayed on the screen of the mobile terminal 270 can try to improve the physical condition by acting according to the advice.

For example, the gait evaluation device 25 may be configured to generate advice that assists the user in decision-making by using a large language models (LLM) system (not illustrated). The LLM system is a system that executes processing using a large language model (not illustrated). The large language model (also referred to as a model) is a deep learning model trained using a large language data set. The LLM system outputs text information corresponding to the content of text information configured in a natural language by using a large language model. The LLM system may be a model capable of inputting and outputting images and sounds. For example, the LLM system is an external system available via an application programming interface (API). The LLM system may be configured to use a dedicated model constructed to perform processing of generating advice that assists the user in decision-making. As long as access can be made from the gait evaluation device 25, the type of the large language model used by the LLM system and a place where the LLM system is arranged are not limited.

### Operation

Next, an example of an operation of the gait evaluation device in the present disclosure will be described with reference to the drawings. The following description of the operation (gait evaluation method) is schematic. Details of the following operation (gait evaluation method) are as described in the above-described configuration.

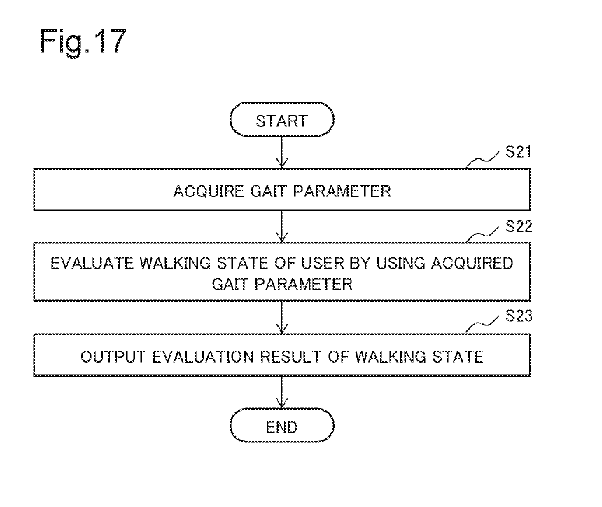

FIG. 17 is a flowchart illustrating an example of the operation of the gait evaluation device in the present disclosure. In the description of the processing as per the flowchart in FIG. 17, a component of the gait evaluation device 25 is assumed as an operating subject. An operating subject of the processing (gait evaluation processing) as per the flowchart of FIG. 17 may be the gait evaluation device 25. For example, evaluation processing is achieved by a processor executing a program stored in a memory mounted in a computer (not illustrated) in which the functions of the gait evaluation device 25 are implemented.

In FIG. 17, the acquisition unit 251 acquires a gait parameter (Step S21).

Then, the evaluation unit 253 evaluates the walking state of the user by using the acquired gait parameter (Step S22).

Then, the output unit 257 outputs an evaluation result of the walking state (Step S23).

As described above, the gait measurement system in the present example embodiment includes the measurement device, the data processing device, and the gait evaluation device. The measurement device includes the sensor that measures sensor data including the acceleration data, the angular velocity data, and the magnetic data. The measurement device is mounted on footwear of the user. The data processing device is the data processing device in the first example embodiment. The gait evaluation device evaluates the physical condition of the user by using the gait parameter calculated by the data processing device.

The gait analysis system in the present example embodiment consistently performs acquisition of the sensor data, rejection of the abnormality data, calculation of the gait parameter, and evaluation of the physical condition of the user. The data processing device rejects the abnormality data included in the sensor data according to the variation in the azimuth angle of the sensor calculated by using the magnetic data. Therefore, according to the present example embodiment, it is possible to detect a sudden direction change or an abnormal motion during walking and to exclude inappropriate data caused by the sudden direction change or the abnormal motion. As a result, according to the present example embodiment, it is possible to achieve a gait analysis system that can be used for health management and exercise assistance of the user.

In one aspect of the present example embodiment, the gait evaluation device generates advice that is optimized according to the physical condition of the user and assists the user in decision-making, by using a machine learning model. The machine learning model is a model constructed by machine learning to output advice according to the physical condition by inputting the gait parameters. The gait evaluation device outputs the generated advice. For example, the advice is displayed on the screen of the mobile terminal used by the user. According to the present aspect, by presenting advice optimized according to the physical condition to the user, it is possible to assist the user in decision-making.

### Third Example Embodiment

Next, a data processing device according to a third example embodiment will be described with reference to the drawings. The data processing device in the present example embodiment has a configuration in which the data processing device in the first and second example embodiments is simplified. For example, the functions of components included in the data processing device in the present example embodiment are achieved by the functions of the components included in the data processing device according to the first and second example embodiments.

### Configuration

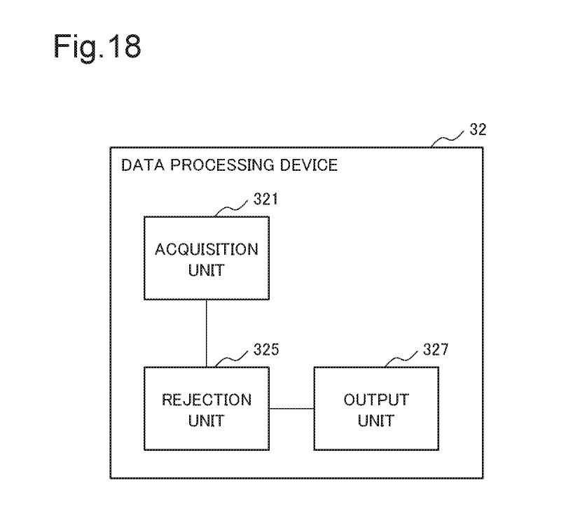

FIG. 18 is a block diagram illustrating an example of a configuration of the data processing device in the present disclosure. A data processing device 32 includes an acquisition unit 321, a rejection unit 325, and an output unit 327.

The acquisition unit 321 acquires sensor data including acceleration data, angular velocity data, and magnetic data measured by the sensor mounted on the footwear of the user. The rejection unit 325 rejects the abnormality data included in the sensor data according to the variation in the azimuth angle of the sensor calculated by using the magnetic data. The output unit 327 outputs the sensor data from which the abnormality data has been excluded.

### Operation

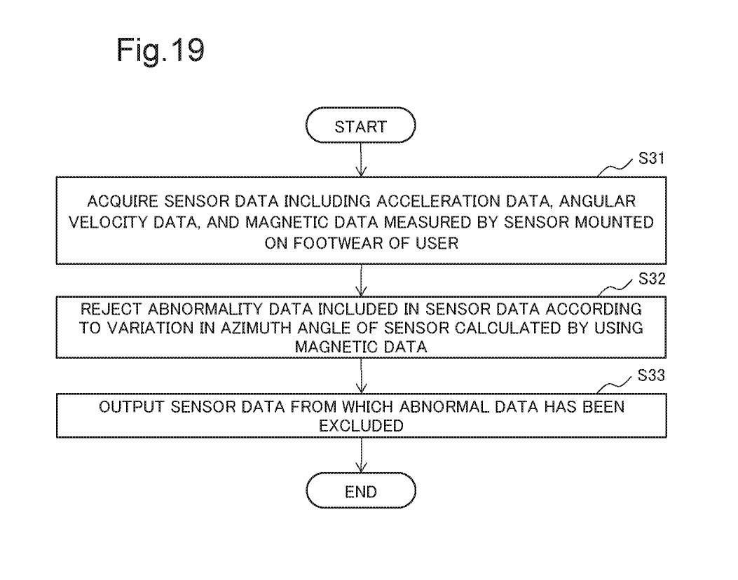

FIG. 19 is a flowchart illustrating an example of an operation of the data processing device in the present disclosure. In the description of the processing as per the flowchart in FIG. 19, a component of the data processing device 32 is assumed as an operating subject. The operating subject of the processing as per the flowchart in FIG. 19 may be the data processing device 32.

In FIG. 19, first, the acquisition unit 321 acquires sensor data including acceleration data, angular velocity data, and magnetic data measured by the sensor mounted on the footwear of the user (Step S31).

Then, the rejection unit 325 rejects the abnormality data included in the sensor data according to the variation in the azimuth angle of the sensor calculated by using the magnetic data (Step S32).

Then, the output unit 327 outputs the sensor data from which the abnormality data has been excluded (Step S33).

In the present example embodiment, the abnormality data included in the sensor data is rejected according to the variation in the azimuth angle of the sensor calculated by using the magnetic data. Therefore, according to the present example embodiment, it is possible to detect a sudden direction change or an abnormal motion during walking and to exclude inappropriate data caused by the sudden direction change or the abnormal motion. Therefore, according to the present example embodiment, it is possible to perform more accurate gait analysis.

### Hardware

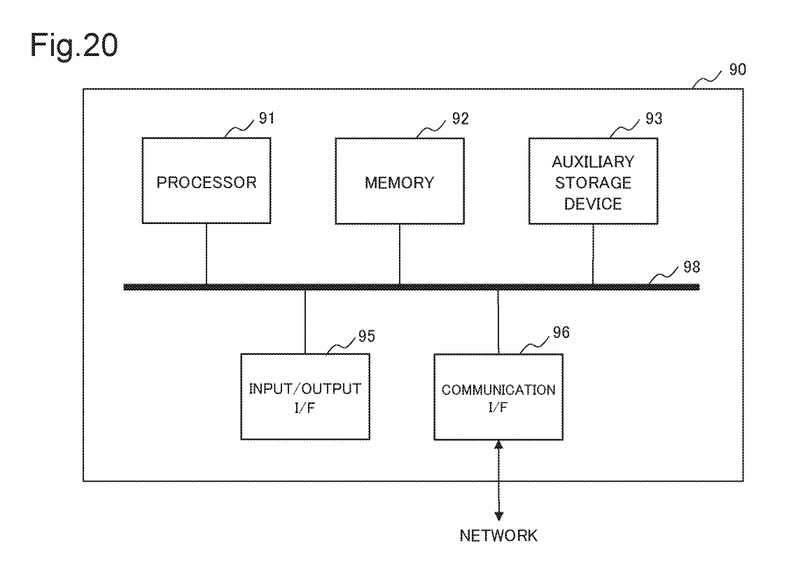

Next, a hardware configuration for executing control and processing in the present disclosure will be described with reference to the drawings. FIG. 20 is a block diagram illustrating an example of a hardware configuration that executes control and processing in the present disclosure. Here, an information processing device 90 (computer) is illustrated as an example of the hardware configuration. The information processing device of FIG. 20 is the configuration example for executing the control and processing in the present disclosure, and does not limit the scope of the present disclosure.

As illustrated in FIG. 20, the information processing device 90 includes a processor 91, a memory 92, an auxiliary storage device 93, an input/output interface 95, and a communication interface 96. In FIG. 20, the interface is abbreviated as an I/F. The information processing device 90 may include a plurality of pieces of at least one of the processor 91, the memory 92, the auxiliary storage device 93, the input/output interface 95, and the communication interface 96. The processor 91, the memory 92, the auxiliary storage device 93, the input/output interface 95, and the communication interface 96 are connected to each other via a bus 98 in such a way that data communication is allowed. The processor 91, the memory 92, the auxiliary storage device 93, and the input/output interface 95 are connected to a network such as the Internet or an intranet via the communication interface 96.

The processor 91 loads a program (command) stored in the auxiliary storage device 93 or the like into the memory 92. For example, the program is a software program for executing the control and processing in the present disclosure. The processor 91 executes the program loaded into the memory 92. The processor 91 executes the control and processing in the present disclosure by executing the program. The processor 91 may be constituted by a single piece of hardware or may be constituted by a plurality of pieces of hardware.

The memory 92 is a storage device having an area into which a program is loaded. A program stored in the auxiliary storage device 93 or the like is loaded into the memory 92 by the processor 91. The memory 92 is achieved by, for example, a volatile memory such as a dynamic random access memory (DRAM). A nonvolatile memory such as a magnetoresistive random access memory (MRAM) may be applied as the memory 92. The memory 92 may be constituted by a single piece of hardware or may be constituted by a plurality of pieces of hardware.

The auxiliary storage device 93 stores various types of data such as programs. For example, the auxiliary storage device 93 is achieved by a local disk such as a hard disk or a flash memory. The auxiliary storage device 93 may be constituted by a single piece of hardware or may be constituted by a plurality of pieces of hardware. The auxiliary storage device 93 may be configured as external hardware. The memory 92 may be formed to store various types of data in such a way that the auxiliary storage device 93 can be omitted.

The input/output interface 95 is an interface for connecting the information processing device 90 and peripheral equipment in accordance with a standard or a specification. The communication interface 96 is an interface for connecting to an external system or device through a network such as the Internet or an intranet in accordance with a standard or a specification. The input/output interface 95 may be constituted by a single piece of hardware or may be constituted by a plurality of pieces of hardware. The input/output interface 95 and the communication interface 96 may be merged as an interface connected to external equipment.

Input equipment such as a keyboard, a mouse, and a touch panel may be connected to the information processing device 90, as necessary. These sorts of input equipment are used to input information and settings. In a case where the touch panel is used as the input equipment, a screen having a touch panel function serves as an interface. The processor 91 and the input equipment are connected via the input/output interface 95.

The information processing device 90 may be provided with display equipment for displaying information. In a case where the display equipment is provided, the information processing device 90 includes a display control device (not illustrated) for controlling display on the display equipment. The information processing device 90 and the display equipment are connected via the input/output interface 95.

The information processing device 90 may be provided with a drive device. The drive device mediates reading of data and a program stored in a recording medium and writing of a processing result of the information processing device 90 to the recording medium between the processor 91 and the recording medium (program recording medium). The information processing device 90 and the drive device are connected via the input/output interface 95.

The above is an example of the hardware configuration for enabling the control and processing in the present disclosure. The hardware configuration of FIG. 20 is an example of the hardware configuration for executing the control and processing in the present disclosure, and does not limit the scope of the present disclosure. A program for causing a computer to execute the control and processing in the present disclosure is also included in the scope of the present disclosure.

A program recording medium in which a program for executing processing in the present example embodiment is recorded is also included in the scope of the present invention. For example, the program recording medium is a computer-readable non-transitory recording medium. The recording medium can be achieved by, for example, an optical recording medium such as a compact disc (CD) or a digital versatile disc (DVD). The recording medium may be achieved by a semiconductor recording medium such as a universal serial bus (USB) memory or a secure digital (SD) card. The recording medium may be achieved by a magnetic recording medium such as a flexible disk, or other recording media.

The components in the present disclosure may be combined in any manner. The components in the present disclosure may be achieved by software. The components in the present disclosure may be achieved by a circuit.

While the present disclosure has been particularly shown and described with reference to example embodiments thereof, the present disclosure is not limited to these example embodiments. It will be understood by those of ordinary skill in the art that various changes in form and details may be made therein without departing from the spirit and scope of the present disclosure as defined by the claims. And each example embodiment can be appropriately combined with other example embodiments.

Some or all of the example embodiments described above may also be described as, but are not limited to, the following Supplementary Notes. In the following Supplementary Notes, dependent items in each category may also depend on other categories. The description included in the following Supplementary Notes has significance as a basis for amendment.

### Supplementary Note 1

A data processing device including:

an acquisition unit that acquires sensor data including acceleration data, angular velocity data, and magnetic data measured by a sensor mounted on footwear of a user;

a rejection unit that rejects abnormality data included in the sensor data according to a variation in an azimuth angle of the sensor calculated by using the magnetic data; and

an output unit that outputs the sensor data from which the abnormality data has been excluded.

### Supplementary Note 2

The data processing device according to Supplementary Note 1, in which

the rejection unit

calculates the azimuth angle of the sensor by using the magnetic data, and

rejects the sensor data measured at a timing at which a variation in the azimuth angle of the sensor is equal to or more than a threshold value.

### Supplementary Note 3

The data processing device according to Supplementary Note 2, in which

the rejection unit

rejects the sensor data measured at a timing at which the azimuth angle of the sensor varies by 90 degrees or more.

### Supplementary Note 4

The data processing device according to Supplementary Note 1, further including:

a calculation unit that calculates a gait parameter quantitatively indicating a walking motion of the user by using the sensor data from which the abnormality data has been excluded, in which

the output unit

outputs the calculated gait parameter.

### Supplementary Note 5

The data processing device according to Supplementary Note 4, in which

the rejection unit

verifies whether the azimuth angle indicates a variation equal to or more than a threshold value, for the sensor data as a calculation source of the gait parameter varying by a reference or more, which has been set in advance.

### Supplementary Note 6

The data processing device according to Supplementary Note 5, in which

the calculation unit

calculates the gait parameter by using the sensor data from which the abnormality data has been excluded.

### Supplementary Note 7

A gait measurement system including:

the data processing device according to Supplementary Note 5 or 6;

a measurement device that is mounted on footwear of a user and includes a sensor that measures sensor data including acceleration data, angular velocity data, and magnetic data; and

a gait evaluation device that evaluates a physical condition of the user by using a gait parameter calculated by the data processing device.

### Supplementary Note 8

The gait measurement system according to Supplementary Note 7,in which

the gait evaluation device

generates advice that is optimized in accordance with the physical condition of the user and assists the user in decision-making, by using a machine learning model that outputs the advice according to the physical condition by inputting the gait parameter, and

outputs the generated advice.

### Supplementary Note 9

A data processing method including:

by a computer,

acquiring sensor data including acceleration data, angular velocity data, and magnetic data measured by a sensor mounted on footwear of a user;

rejecting abnormality data included in the sensor data according to a variation in an azimuth angle of the sensor calculated by using the magnetic data; and

outputting the sensor data from which the abnormality data has been excluded.

### Supplementary Note 10

A program for causing a computer to execute a process including:

acquiring sensor data including acceleration data, angular velocity data, and magnetic data measured by a sensor mounted on footwear of a user;

rejecting abnormality data included in the sensor data according to a variation in an azimuth angle of the sensor calculated by using the magnetic data; and

outputting the sensor data from which the abnormality data has been excluded.

Some or all of the configurations described in Supplementary Notes 2 to 8 dependent on the above-described Supplementary Note 1 can also be dependent on Supplementary Notes 9 and 10 by a dependency relationship similar to that of Supplementary Notes 2 to 8. Some or all of the configurations described as the Supplementary Notes can be similarly dependent on not only the Supplementary Notes 1, 9, and 10, but also diverse pieces of hardware and software, various recording means for recording software, or systems without departing from the above-described example embodiments.

1. A data processing device comprising:
a memory storing instructions; and
a processor connected to the memory and configured to execute the instructions to:
acquire, from an inertial measurement unit and a magnetic sensor mounted on footwear worn by a user, time-series sensor data representing movement of a foot of the user, the sensor data including acceleration data, angular velocity data, and magnetic data based on geomagnetism;
determine, from the magnetic data, an azimuth angle of the magnetic sensor relative to a geographical direction;
identify, based on a variation of the determined azimuth angle exceeding a predetermined threshold, a period during which the sensor data is indicative of a change in a traveling direction of the user;
reject abnormality data included in the sensor data, the abnormality data corresponding to the identified period and being caused by the change in the traveling direction or an abnormality in movement of the foot; and
output the sensor data from which the abnormality data has been excluded for use in subsequent gait analysis for the user.

2. The data processing device according to claim 1, wherein
the processor is configured to execute the instructions to:
determine the azimuth angle at each of a plurality of timings included in the time-series sensor data;
calculate a variation in the azimuth angle between azimuth angles corresponding to pieces of magnetic data that are temporally continuous; and
identify, as the period, a time zone in which the variation in the azimuth angle is equal to or more than the predetermined threshold.

3. The data processing device according to claim 2, wherein
the predetermined threshold is set such that the period includes a time zone in which the azimuth angle of the magnetic sensor varies by 90 degrees or more.

4. The data processing device according to claim 1, wherein
the processor is configured to execute the instructions to:
calculate, from the sensor data from which the abnormality data has been excluded, a gait parameter quantitatively indicating a walking state of the user; and
output the calculated gait parameter.

5. The data processing device according to claim 4, wherein
the processor is configured to execute the instructions to:
verify whether a variation in the azimuth angle is equal to or more than a threshold value in a case where a change in the gait parameter between gait parameters calculated for gait cycles that are temporally continuous is equal to or more than a predetermined reference value.

6. The data processing device according to claim 5, wherein
the processor is configured to execute the instructions to:
calculate the gait parameter using the sensor data from which the abnormality data has been excluded.

7. A gait measurement system comprising:
the data processing device according to claim 5;
a measurement device that is mounted on footwear worn by a user and includes an inertial measurement unit and a magnetic sensor configured to measure, as the time-series sensor data, acceleration data, angular velocity data, and magnetic data based on geomagnetism; and
a gait evaluation device comprising a memory storing instructions and a processor configured to execute the instructions to evaluate a walking state of the user using a gait parameter calculated by the data processing device.

8. The gait measurement system according to claim 7, wherein
the processor of the gait evaluation device is configured to execute the instructions to:
generate advice information that is optimized in accordance with the physical condition of the user and assists the user in decision making, by using a machine learning model that outputs the advice information based on an input including the gait parameter; and
output the generated advice information that assists the user in decision making according to the physical condition of the user to a terminal used by the user.

9. A data processing method comprising:
by a computer,
acquiring, from an inertial measurement unit and a magnetic sensor mounted on footwear worn by a user, time-series sensor data representing movement of a foot of the user, the sensor data including acceleration data, angular velocity data, and magnetic data based on geomagnetism;
determining, from the magnetic data, an azimuth angle of the magnetic sensor relative to a geographical direction;
identifying, based on a variation of the determined azimuth angle exceeding a predetermined threshold, a period during which the sensor data is indicative of a change in a traveling direction of the user;
rejecting abnormality data included in the sensor data, the abnormality data corresponding to the identified period and being caused by the change in the traveling direction or an abnormality in movement of the foot; and
outputting the sensor data from which the abnormality data has been excluded for use in subsequent gait analysis for the user.

10. A non-transitory computer-readable recording medium storing a program for causing a computer to execute a process comprising:
acquiring, from an inertial measurement unit and a magnetic sensor mounted on footwear worn by a user, time-series sensor data representing movement of a foot of the user, the sensor data including acceleration data, angular velocity data, and magnetic data based on geomagnetism;
determining, from the magnetic data, an azimuth angle of the magnetic sensor relative to a geographical direction;
identifying, based on a variation of the determined azimuth angle exceeding a predetermined threshold, a period during which the sensor data is indicative of a change in a traveling direction of the user;
rejecting abnormality data included in the sensor data, the abnormality data corresponding to the identified period and being caused by the change in the traveling direction or an abnormality in movement of the foot; and
outputting the sensor data from which the abnormality data has been excluded for use in subsequent gait analysis for the user.

11. The data processing method according to claim 9, wherein
determining the azimuth angle and identifying the period comprise:
determining the azimuth angle at each of a plurality of timings included in the time-series sensor data;
calculating a variation in the azimuth angle between azimuth angles corresponding to pieces of magnetic data that are temporally continuous; and
identifying, as the period, a time zone in which the variation in the azimuth angle is equal to or more than the predetermined threshold.

12. The data processing method according to claim 11, wherein
the predetermined threshold is set such that the period includes a time zone in which the azimuth angle of the magnetic sensor varies by 90 degrees or more.

13. The data processing method according to claim 9, further comprising:
calculating, from the sensor data from which the abnormality data has been excluded, a gait parameter quantitatively indicating a walking state of the user; and
outputting the calculated gait parameter.

14. The data processing method according to claim 13, further comprising:
verifying whether a variation in the azimuth angle is equal to or more than a threshold value in a case where a change in the gait parameter between gait parameters calculated for gait cycles that are temporally continuous is equal to or more than a predetermined reference value.

15. The data processing method according to claim 14, wherein
calculating the gait parameter comprises calculating the gait parameter using the sensor data from which the abnormality data has been excluded.

16. The non-transitory computer-readable recording medium according to claim 10, wherein
the process further comprises:
determining the azimuth angle at each of a plurality of timings included in the time-series sensor data;
calculating a variation in the azimuth angle between azimuth angles corresponding to pieces of magnetic data that are temporally continuous; and
identifying, as the period, a time zone in which the variation in the azimuth angle is equal to or more than the predetermined threshold.

17. The non-transitory computer-readable recording medium according to claim 16, wherein
the predetermined threshold is set such that the period includes a time zone I n which the azimuth angle of the magnetic sensor varies by 90 degrees or more.

18. The non-transitory computer-readable recording medium according to claim 10, wherein
the process further comprises:
calculating, from the sensor data from which the abnormality data has been excluded, a gait parameter quantitatively indicating a walking state of the user; and
outputting the calculated gait parameter.

19. The non-transitory computer-readable recording medium according to claim 18, wherein
the process further comprises:
verifying whether a variation in the azimuth angle is equal to or more than a threshold value in a case where a change in the gait parameter between gait parameters calculated for gait cycles that are temporally continuous is equal to or more than a predetermined reference value.

20. The non-transitory computer-readable recording medium according to claim 19, wherein
calculating the gait parameter comprises calculating the gait parameter using the sensor data from which the abnormality data has been excluded.
